(19) United States
(12) Patent Application Publication
(10) Pub. No.: US 2024/0360516 A1
(43) Pub. Date: Oct. 31, 2024

(54) METHODS FOR THE ANALYSIS OF GENE EXPRESSION AND USES THEREOF

(71) Applicant: Peter MacCallum Cancer Institute, Melbourne, VIC (AU)

(72) Inventors: Sarah-Jane DAWSON, Victoria (AU); Mark Agnel Frederick DAWSON, Victoria (AU); Sandunie Dineika CHANDRANANDA, Victoria (AU); Paul Sung-Hao YEH, Victoria (AU)

(21) Appl. No.: 18/285,435

(22) PCT Filed: Apr. 5, 2022
(86) PCT No.: PCT/AU2022/050301

§ 371 Date: Oct. 3, 2023

(30) Foreign Application Priority Data
Apr. 8, 2021 (AU) 2021901031

(51) Int. Cl. C12Q 1/6886; C12Q 1/6869

(52) U.S. Cl. CPC C12Q 1/6886; C12Q 1/6869; C12Q 2600/112; C12Q 2600/158; G01N 2800/52

(57) ABSTRACT

The present disclosure relates generally to methods for the analysis of gene expression. In particular, the methods of the present disclosure are based on the measurement of gene expression from fragments of cell-free DNA (cfDNA), which is useful in non-invasive methods for monitoring disease status in cancer patients and in methods for the treatment of cancer patients.

### RELATED APPLICATIONS

This application claims priority from Australian Provisional Application No. 2021901031, filed on 8 Apr. 2021, the entire contents of which is hereby incorporated by reference.

### FIELD

The present disclosure relates generally to methods for the analysis of gene expression. In particular, the methods of the present disclosure are based on the measurement of gene expression from fragments of cell-free DNA (cfDNA), which is useful in non-invasive methods for monitoring disease status in cancer patients and in methods for the treatment of cancer patients.

### BACKGROUND

Cancer management is in an era of precision medicine, however, the key to improving clinical outcomes is a better understanding of cancer evolution within each patient, especially in the context of therapy. Much effort has been made to characterise genomic evolution, however, there is growing evidence that non-genomic evolution involving transcriptional adaptation plays an equally important role. In this respect, the ability to understand these changes is greatly limited by technical challenges, not to mention the invasive nature of serial tissue biopsy. As such, the use of non-invasive approaches to obtain non-genomic information is highly attractive.

The use of cell-free tumour DNA (ctDNA) has revolutionised cancer detection and monitoring and given great insights into the genomic changes that occur during cancer therapy. Recent studies have utilised plasma ctDNA in cancer patients to develop methods to infer gene expression profiles of the tumour (Ulz et al., 2016, Nature Genetics, 48 (10): 1273-1278). The premise of such methods is that ctDNA fragmentation is non-random and closely linked with nucleosomal architecture. Notably, the region surrounding the transcriptional start sites (TSS) of actively expressed genes are under-represented in ctDNA, as without the protection of the nucleosome, these fragments are rapidly digested by plasma nucleases. Therefore, mapping ctDNA fragments through whole-genome sequencing (WGS) generates nucleosome occupancy maps that allows for a binary determination of gene expression (i.e., expressed or unexpressed).

Although a highly promising tool, the major limitation of previously developed methods is that high tumour purity is required to obtain such information, as much of the cell-free DNA (cfDNA) in plasma is derived from normal (i.e., non-tumour) haematopoietic cells. As a result, it has been suggested that such previously developed methods are only useful for the binary assessment of gene expression for genes located in genomic regions with high-amplitude copy number amplifications (Ulz et al., 2016, supra). Hence, this form of analysis is not sensitive or accurate enough to measure dynamic changes in gene expression across all genomic regions, independent of amplitude of copy number amplification. Accordingly, there is a need to develop methods for the measurement of gene expression using cfDNA with graded gene expression predictions in plasma, and to provide dynamic assessment of gene expression over time, such as to reflect the changes in gene expression that occur in patients before, during and after treatment for cancer.

### SUMMARY

In one aspect, the present disclosure provides a method for determining the level of gene expression from cell-free DNA (cfDNA), the method comprising:
a. providing a sample obtained from a subject, wherein the sample comprises fragments of cfDNA;
b. generating a plurality of sequence reads by sequencing the fragments of cfDNA, wherein the resulting plurality of sequence reads correspond to fragments of cfDNA of variable lengths;
c. aligning the plurality of sequence reads generated in step (b) with a reference genome;
d. identifying sets of sequence reads from step (c) that align to genomic regions of the reference genome comprising a gene transcriptional start site (TSS), wherein each set of sequence reads aligned to a genomic region of the reference genome corresponds to a single gene;
e. determining the read depth of the sets of sequence reads identified in step (d), wherein read depth is the number of unique sequence reads that align to a genomic region;
f. generating a corrected read depth based on the read depth determined in step (e), a flanking region read depth and the total number of sequence reads in the sample, wherein the flanking region read depth is the number of unique sequence reads that align with the flanking region, wherein the flanking region comprises the centre of the nucleosome depleted region (NDR) for each TSS +/−5000 base pairs (bp); and
g. generating a plurality of gene expression categories by ranking the corrected read depth for each genomic region from the lowest corrected read depth to highest corrected read depth, and generating gene expression categories that correspond to the level of gene expression for each gene based on the ranked corrected read depth, wherein the genes with the lowest corrected read depth are grouped into high gene expression categories, and the genes with the highest corrected read depth are grouped into a low gene expression categories.

In another aspect, the present disclosure provides a method for determining a level of gene expression from cfDNA, the method comprising:
a. providing a sample obtained from a subject, wherein the sample comprises fragments of cfDNA;
b. generating a plurality of sequence reads by sequencing the fragments of cfDNA, wherein the resulting plurality of sequence reads correspond to fragments of cfDNA of variable lengths;
c. aligning the plurality of sequence reads generated in step (b) with a reference genome;
d. identifying sets of sequence reads from step (c) that align to genomic regions of the reference genome comprising a gene TSS, wherein each set of sequence reads aligned to a genomic region of the reference genome corresponds to a single gene;
e. determining the read depth of the sets of sequence reads identified in step (d), wherein read depth is the number of unique sequence reads that align to each genomic region; and
f. generating a corrected read depth based on the read depth determined in step (e), a flanking region read depth and the total number of sequence reads in the sample, wherein the flanking region read depth is the number of unique sequence reads that align with the flanking region, wherein the flanking region comprises the centre of the NDR for each TSS +/−5000 bp, wherein the corrected read depth corresponds to the level of gene expression for each gene, wherein the genes with the lowest corrected read depth have the highest gene expression, and wherein the genes with the highest corrected read depth have the lowest gene expression.

In another aspect, the present disclosure provides a method for determining the likelihood that a subject has cancer, the method comprising:
a. providing a sample obtained from the subject, wherein the sample comprises fragments of cfDNA;
b. determining the level of gene expression of one or more genes in the sample according to the method as disclosed herein;
c. comparing the level of gene expression determined in step (b) with a reference level of gene expression for the one or more genes; and
d. based on the comparison in step (c) determining the likelihood that the subject has cancer.

In another aspect, the present disclosure provides a method for the treatment of a subject with cancer, the method comprising:
a. providing a sample obtained from the subject, wherein the sample comprises fragments of cfDNA;
b. determining the likelihood that a subject has cancer according to the method as disclosed herein; and
c. where based on the determination in step (b) the subject has a high likelihood of having cancer, treating the subject with a treatment for said cancer.

In another aspect, the present disclosure provides a method for monitoring disease status in a subject having cancer, the method comprising:
a. providing a first sample obtained from the subject, wherein the first sample comprises fragments of cfDNA;
b. determining the level of gene expression of one or more genes in the first sample according to the method as disclosed herein;
c. repeating step (b) with one or more additional samples obtained from the subject at a subsequent time point(s);
d. determining the tumour fraction (%) for the first sample and the one or more additional samples;
e. normalising the level of gene expression in each sample determined in steps (b) and (c) based on tumour fraction; and
f. comparing the normalised level of gene expression for each gene in the first sample with the normalised level of gene expression for each gene in the one or more additional samples to evaluate whether there has been a change in gene expression over time.

### BRIEF DESCRIPTION OF THE DRAWINGS

Embodiments of the disclosure are described herein, by way of non-limiting example only, with reference to the accompanying drawings.

FIG. 1 shows that gene expression measured from cell-free DNA (cfDNA) correlates highly with bone marrow RNA-sequencing (RNA-seq). (A) A schematic representation of the method for determining gene expression from plasma-derived cfDNA as compared to standard RNA-seq methods from bone marrow-derived RNA. (B) Graphical representations of the most highly expressed genes are determined from matched bone marrow RNA-seq represented as density (y-axis) and expression level of genes (TMM-normalised fragment per kilobase million (FPKM), top panel); and relative sequence coverage (y-axis) and position relative to the transcription start site (TSS) (bp; x-axis) in a baseline pre-treatment plasma sample from patient AZA008 with myelodysplastic syndrome (MDS) from the most highly expressed genes to the least expressed genes (bottom panel). (C) A graphical representation of RNA-seq expression values (TMM-normalised FPKM; y-axis) and predicted gene expression from cfDNA sequencing quantified into ranked levels (x-axis). Statistical significance of the difference between genes in all lower ranks against rank 1 genes (most highly expressed) was quantified by pairwise Wilcoxon rank-sum tests (*: p<=0.05, **: p<=0.01, ***: p<=0.001, ****: p<=0.0001). (D) A schematic representation of the patient cohorts described elsewhere herein, including the number of plasma samples for whole-genome sequencing from patients with different cancer types compared to plasma samples from healthy volunteers. (E) A graphical representation of the clustering of MDS and AML patients compared to healthy volunteers using principal component analysis (PCA) of gene expression as measured from cfDNA according to the methods described herein (top panel). These values were plotted as a discriminatory variable and expressed as a receiver operating characteristic (ROC) curve comparing cancer vs healthy (bottom panel). (F) A graphical representation of the clustering of breast cancer (Cohort 1), lung cancer and melanoma compared patients compared to healthy volunteers using PCA of gene expression as measured from cfDNA according to the methods described herein (top panel). These values were plotted as a discriminatory variable and expressed as a ROC curve comparing cancer vs healthy (bottom panel). (G) A graphical representation of the clustering of breast cancer patients (Cohort 2), using PCA of gene expression as measured from cfDNA according to the methods described herein (top panel). These values were plotted as a discriminatory variable and expressed as a ROC curve comparing cancer vs healthy (bottom panel).

FIG. 2 shows that gene expression measured from cfDNA correlates highly with matched bone marrow RNA-seq and detects tumour-specific gene expression profiles. (A) A graphical representation of plasma cfDNA-inferred gene expression (plasma expression (CPMᵢ); y-axis) from the top 500 most highly and least expressed genes (x-axis) as determined in matched RNA-seq data, ****p≤0.0001. (B) A graphical representation of plasma cfDNA-inferred gene expression (plasma expression (CPMᵢ); y-axis) of expressed or non-expressed genes (x-axis), ****p≤0.0001. (C) A series of graphical representations of the prediction accuracy (%; y-axis) for the top 500 (left panel), 1000 (middle panel) and 5000 (right panel) most highly and least expressed genes determined from the pre-treatment plasma samples of MDS patients and usable whole genome coverage depths (x-axis). (D) A graphical representation of RNA-seq expression values (TMM-normalised FPKM; y-axis) and predicted gene expression from cfDNA sequencing quantified into ranked levels (x-axis). Statistical significance of the difference between genes in all lower ranks against rank 1 genes (most highly expressed) was quantified by pairwise Wilcoxon rank-sum tests (*: p≤0.05, **: p≤0.01, ***: p≤0.001, ****: p≤0.0001) (E) A series of graphical representations of the PCA of unsupervised clustering for MDS, breast cancer, melanoma and lung cancer, together with healthy controls.

FIG. 3 shows the gradient of variation of read depth from the most highly and least expressed genes. A series of graphical representations of read depth (relative coverage; y-axis) across genomic regions comprising the TSS +/−1000 bp with respect to the TSS (position relative to TSS (bp); x-axis) in pre-treatment plasma samples from MDS patients (A)-(I) as determined from the deciles of matched bone marrow RNA-seq count distribution.

FIG. 4 shows the prediction accuracy of the plasma cfDNA method using the NDR and 2K_TSS genomic regions. A series of graphical representation of accuracy (%; y-axis) and whole genome coverage depths (usable WGS coverage; x-axis) for the top (A) 500, (B) 1000 and (C) 5000 most highly and least expressed genes from pre-treatment plasma samples from MDS patients.

FIG. 5 shows the prediction accuracy of the Ulz et al. method using the NDR and the wider 2K_TSS genomic regions. A series of graphical representation of accuracy (%; y-axis) and whole genome coverage depths (usable WGS coverage; x-axis) for the top (A) 500, (B) 1000 and (C) 5000 most highly and least expressed genes from pre-treatment plasma samples from MDS patients.

FIG. 6 shows a comparative analysis of the prediction accuracy of the plasma cfDNA method and the Ulz et al. method. (A) A graphical representation of accuracy (%; y-axis) and whole genome coverage depths (usable WGS coverage; x-axis) for the top (A) 500, (B) 1000 and (C) 5000 most highly and least expressed genes from pre-treatment plasma samples from MDS patients.

FIG. 7 shows a comparative analysis of RNA-seq counts of genes defined as comprising the NDR or wider 2K_TSS genomic regions. (A) A series of graphical representations of RNA-seq counts (logged RNA-seq counts (FPMK); y-axis) and the decile rank of genes defined as comprising the NDR region (x-axis) from pre-treatment plasma samples from MDS patients, *p≤0.05; **p≤0.01; ***p≤0.001; ****p≤0.0001; ns=non-significant using one-way Wilcoxon rank-sum tests. (B) A series of graphical representations of RNA-seq counts (logged RNA-seq counts (FPMK); y-axis) and the decile rank of genes defined as comprising the wider 2K_TSS genomic region (x-axis) from pre-treatment plasma samples from MDS patients, *p≤0.05; **p≤0.01; ***p≤0.001; ****p≤0.0001; ns=non-significant using one-way Wilcoxon rank-sum tests.

FIG. 8 shows that malignant tumour types can be separated by estimated tumour fraction. A series of graphical representations of PCA of unsupervised clustering between the estimated tumour fraction based on the plasma cfDNA-generated corrected read depth of genes defined as genomic regions comprising the NDR from plasma samples from (A) MDS, (B) breast cancer, (C) melanoma and (D) lung cancer patients as compared to healthy controls.

FIG. 9 shows that malignant tumour types can be separated by estimated tumour fraction. A series of graphical representations of PCA of unsupervised clustering between the estimated tumour fraction based on the plasma cfDNA-generated corrected read depth of genes defined as genomic regions comprising 2K_TSS from plasma samples from (A) MDS, (B) breast cancer, (C) melanoma and (D) lung cancer patients as compared to healthy controls.

FIG. 10 shows that gene expression measured from plasma cfDNA allows for characterisation of cancer pathways. (A) A graphical representation of the gene expression of the top 500 over-expressed genes in all cancer samples compared to plasma from healthy controls. (B) A graphical representation of a pathway enrichment analysis of the top 500 over-expressed genes showing enrichment (x-axis) of HALLMARK pathways relating to proliferation (y-axis). (C) A schematic representation of heat maps comparing the enrichment scores of HALLMARK pathways in proliferation, DNA damage, signalling and immune subsets for different solid malignancies, *=top 5 pathways enriched within each cancer type. (D) A series of graphical representations of enrichment (x-axis) of top 5 HALLMARK pathways (y-axis) for breast cancer (left panel), lung cancer (middle panel), and melanoma (right panel). (E) A graphical representation of the supervised clustering of the top filtered genes from all solid malignancy plasma samples by PCA demonstrating separation of breast cancer, lung cancer and melanoma. (F) A series of graphical representations of the supervised clustering of cancer-specific gene sets from RNA-seq data for solid malignancies from the TCGA by PCA demonstrating separation of breast invasive carcinoma (BRCA), lung adenocarcinoma (LUAD) and skin cutaneous melanoma (SKCM) samples.

FIG. 11 shows that gene expression measured from plasma cfDNA facilitates non-invasive, serial monitoring of transcriptional evolution following cancer therapy. (A) A graphical representation of pathway enrichment analysis of plasma samples analysed by RNA-seq (left panel) and the plasma cfDNA method (right panel) at the time of progression in MDS patients undergoing azacitidine therapy. (B) A schematic representation of heat maps comparing the enrichment scores of HALLMARK pathways in proliferation, DNA damage, signalling and immune subsets for matched RNA-seq and plasma cfDNA-derived gene expression at the time of progression in MDS patients undergoing azacitidine therapy, *=top 5 pathways enriched within each transcriptome source. (C) A graphical representation of the top pathways upregulated (y-axis) at the time of progression in breast cancer patients undergoing CDK4/6 inhibitor therapy. (D) A graphical representation of gene expression changes from baseline to progression in breast cancer patients undergoing CDK4/6 inhibitor therapy. Annotated genes represent genes that were also found to be upregulated in the NeoPalAna clinical trial, described elsewhere herein. (E) A graphical representation of tumour fraction (%; y-axis) between pre-treatment plasma samples and progression plasma samples in breast cancer patients undergoing CDK4/6 inhibitor therapy. (F) A graphical representation of the top pathways upregulated (y-axis) at the time of progression in melanoma patients undergoing MAPK inhibitor therapy. (G) A graphical representation of gene expression changes from baseline to progression in melanoma patients undergoing MAPK inhibitor therapy. Annotated genes represent genes that were also found to be upregulated in Hugo et al. (2015, Cell, 162:1271-1285). (H) A graphical representation of tumour fraction (%; y-axis) between pre-treatment plasma samples and progression plasma samples in melanoma patients undergoing MAPK inhibitor therapy. (I) A schematic representation of heat maps comparing the enrichment scores of HALLMARK pathways in proliferation, DNA damage, signalling and immune subsets at the time of progression in patients with MDS, breast cancer and melanoma, *=top 5 pathways enriched within each tumour type.

FIG. 12 shows that gene expression measured from plasma cfDNA can uncover transcriptional adaptation resulting in treatment resistance. (A) A schematic representation of the treatment of AML patient 2 with a BET inhibitor, molibresib. (B) A graphical representation of the copy number profiles of AML patient 2 at baseline (top panel) and progression (bottom panel) generated by plasma DNA sequencing. (C) A graphical representation of single cell transcriptomes of the top 10% expressed genes at baseline and relapse from individual bone marrow blast cells from AML patient 2 using scRNA sequencing (left panel) and plasma cfDNA-derived gene expression (right panel). (D) A graphical representation of the change in expression from baseline to progression (CPM; y-axis) in the leukaemia stem cell (LSC) gene expression signature in AML patient 2. Black bars highlight genes that were also expressed in more cells at progression from the scRNA-seq data. (E) A schematic representation of a patient with EGFR mutant non-small cell lung cancer (NSCLC) undergoing EGFR tyrosine kinase therapy (i.e., erlotinib followed by osimertinib). (F) A graphical representation of the copy number profiles of the NSCLC patient at baseline (top panel) and progression (bottom panel) generated by plasma DNA sequencing. (G) A photographic representation of histology and immunohistochemistry of a liver biopsy taken from the NSCLC patient at the time of progression, which demonstrates small cell transformation, scale bar represents 100 μm. (H) A graphical representation of the change in expression from baseline to progression (CPM; y-axis) in the neuroendocrine gene expression signature in the NSCLC patient. Black bars highlight genes that were also shown to be expressed by immunohistochemistry of the liver biopsy showing small cell transformation.

FIG. 13 shows a comparison of single cell transcriptome generated by scRNA sequencing and gene expression measured from plasma cfDNA. (A) A graphical representation of the single cell transcriptomes of the top 10% expressed genes at baseline, remission and relapse from individual bone marrow blast cells from AML patient 1 using scRNA. (B) A graphical representation of the single cell transcriptomes of the top 10% expressed genes at baseline, remission and relapse from individual bone marrow blast cells from AML patient 1 using plasma cfDNA-derived gene expression.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a treatment” includes a single treatment, as well as two or more treatments; and so forth.

In the context of this specification, the term “about” is understood to refer to a range of numbers that a person of skill in the art would consider equivalent to the recited value in the context of achieving the same function or result.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The present disclosure is predicated, in part, on the surprising finding that cell-free DNA (cfDNA) may be used to accurately determine gene expression. Further, it has been shown that the methods disclosed herein determine the level of gene expression from cfDNA with a high degree of concordance to standard RNA sequencing (RNA-seq) methods. These findings have been reduced to practice in methods for determining the level of gene expression from cfDNA, methods for the determining the likelihood that a subject has cancer, methods for the treatment of cancer, and methods for monitoring disease status by providing dynamic assessment of gene expression in patients before, during and/or after treatment for cancer. The methods described herein thereby provide a minimally invasive technique to concurrently capture genomic and non-genomic changes that occur over time, which is sensitive enough to accurately predict disease recurrence, in some instances prior to the presentation of clinicopathological features.

### Methods for Determining Gene Expression

Accordingly, in an aspect, the present disclosure provides a method for determining a level of gene expression from cfDNA, the method comprising:
a. providing a sample obtained from a subject, wherein the sample comprises fragments of cfDNA;
b. generating a plurality of sequence reads by sequencing the fragments of cfDNA, wherein the resulting plurality of sequence correspond to fragments of cfDNA of variable lengths;
c. aligning the plurality of sequence reads generated in step (b) with a reference genome;
d. identifying sets of sequence reads from step (c) that align to genomic regions of the reference genome comprising a gene transcriptional start site (TSS), wherein each set of sequence reads aligned to a genomic region of the reference genome corresponds to a single gene;
e. determining the read depth of the sets of sequence reads identified in step (d), wherein read depth is the number of unique sequence reads that align to each genomic region; and
f. generating a corrected read depth based on the read depth determined in step (e), a flanking region read depth and the total number of sequence reads in the sample, wherein the flanking region read depth is the number of unique sequence reads that align with the flanking region, wherein the flanking region comprises the centre of the nucleosome depleted region (NDR) for each TSS +/−5000 base pairs (bp), wherein the corrected read depth corresponds to the level of gene expression for each gene, wherein the genes with the lowest corrected read depth have the highest gene expression, and wherein the genes with the highest corrected read depth have the lowest gene expression.

The terms “cell-free DNA” or “cfDNA” refer to fragments of extracellular DNA that originates from cell death and cellular proliferation and circulate in peripheral blood. cfDNA is typically fragmented to an average length of about 140 to 170 bp.

In an embodiment, the cfDNA comprises “circulating tumour DNA” or “ctDNA”.

It would be known to persons skilled in the art that ctDNA has previously been used to detect and monitor genomic changes that occur during cancer disease progression and therapy as described by, for example, Wan et al. (2017, Nature Reviews Cancer, 17 (4): 223-238).

In an embodiment, the cfDNA comprises circulating tumour DNA (ctDNA).

In an embodiment, fragments of cfDNA are isolated from a sample obtained from a subject.

In an embodiment, the sample is selected from the group consisting of whole blood, serum and plasma. In an exemplary embodiment, the sample is plasma.

Methods for the extraction and processing of whole blood, or a fraction of whole blood, such as serum and plasma, would be known to persons skilled in the art, illustrative examples of which include the standard operative procedures for serum and blood collection described by Tuck et al. (2009, Journal of Proteome Research, 8 (1): 113-117).

The term “cancer” as used herein means any condition associated with aberrant cell proliferation. Such conditions will be known to persons skilled in the art.

In an embodiment, the cancer is a haematological malignancy. In another embodiment, the haematological malignancy is a myeloid neoplasm. In yet another embodiment, the haematological malignancy is selected from the group consisting of myelodysplastic syndrome (MDS) and acute myeloid leukaemia (AML).

MDS is a clonal haematopoietic stem cell disorder of the bone marrow characterised by peripheral blood cytopenias, excess of blasts and high-risk of progression to AML.

AML is characterised by the malignant expression of immature haematopoietic cells and can arise de novo, or as part of invariable progression from MDS.

In an embodiment, the methods disclosed herein are also useful in the detection of non-genomic changes in cfDNA derived from solid tumours.

Thus, in an embodiment, the cancer is a solid tumour. In another embodiment, the solid tumour is selected from the group consisting of breast cancer, lung cancer and melanoma.

The subject may be a human or a mammal of economic importance and/or social importance to humans, for instance, carnivores other than humans (e.g., cats and dogs), swine (e.g., pigs, hogs, and wild boars), ruminants (e.g., cattle, oxen, sheep, giraffes, deer, goats, bison, and camels), horses, and birds including those kinds of birds that are endangered, kept in zoos, and fowl, and more particularly domesticated fowl, e.g., poultry, such as turkeys, chickens, ducks, geese, guinea fowl, and the like, as they are of economic importance to humans. The term “subject” does not denote a particular age. Thus, adult, juvenile and new born subjects are intended to be covered.

The terms “subject”, “individual” and “patient” are used interchangeably herein to refer to any subject to which the present disclosure may be applicable. In an embodiment, the subject is a mammal. In another embodiment, the subject is a human.

The present disclosure is predicated, at least in part, on the finding that the fragmentation of cfDNA is non-random and is related to nucleosome organisation and architecture. In particular, the region surrounding the TSS of highly expressed genes shows periodic oscillations in read depth (i.e., coverage) in comparison to unexpressed genes, in addition to a distinct drop in coverage in the region ˜150 base pairs (bp) upstream of the TSS and ˜50 bp downstream of the TSS.

Nucleosomes play an essential role in the management of the eukaryotic genome by facilitating its packaging inside the nucleus. They also regulate basic genomic processes such as transcription, replication and recombination, either directly by controlling the physical access of regulators to DNA or indirectly by modulating their binding through a complex repertoire of histone modifications.

Structurally, the nucleosome is defined as the basic structural repeat unit of chromatin. It is composed of a nucleosome core containing 147 bp of DNA wrapped around a central histone octamer containing two molecules each of the four core histones (H2A, H2B, H3 and H4) and a “linker” DNA (i.e., a “DNA linker sequence”) of characteristic length, which connects one nucleosome to the next. A single molecule of histone H1 (linker histone) is bound to the nucleosome at the point where the DNA enters and exits the core, and to the linker DNA. The DNA within the nucleosome core is protected from nucleases by the core histones, whereas the linker DNA is vulnerable to digestion.

In an embodiment, each sequence read corresponds to a fragment of cfDNA comprising one or both of: (i) at least one nucleosome; and (ii) all or part of a DNA linker sequence adjacent to the at least one nucleosome.

The number of nucleosomes in each corresponding fragment cfDNA may be inferred from the length of the sequence read, which correspond to a fragment of cfDNA in the sample. For example: (i) sequence reads of 170 bp to 250 bp in length most likely comprise one nucleosome and part of the DNA linker sequence adjacent to the nucleosome; (ii) sequence reads of 250 bp to 350 bp in length most likely comprise at least two nucleosomes and all of the DNA linker sequence adjacent to the nucleosomes; (iii) sequence reads of 350 bp to 550 bp in length most likely comprise at least three nucleosomes and all or part of the DNA linker sequence adjacent to the nucleosomes.

In an embodiment, the aligned sequence reads are trimmed to remove all or part of the DNA linker sequence.

In an embodiment, the alignment of the trimmed sequence reads to the reference genome is adjusted to generate a new start coordinate. In accordance with this embodiment, the new start coordinate is generated with reference to the estimated number of nucleosomes comprised in each sequence read, as described elsewhere herein. For example, (i) for sequence reads between 80 bp to 169 bp in length, the new start coordinate of the of the trimmed sequence read=original start coordinate+(sequence read length−50 bp)/2; (ii) for sequence reads between 170 bp to 250 bp in length, the new start coordinate of the of the trimmed sequence read=original start coordinate+(sequence read length−50 bp)/2.5; (iii) for sequence reads 250 bp to 350 bp in length, the new start coordinate of the of the trimmed sequence read=original start coordinate+(sequence read length−50 bp)/3; and (iv) for sequence reads 350 bp to 550 bp in length, the new start coordinate of the of the trimmed sequence read=original start coordinate+(sequence read length−50 bp)/4.

In an embodiment, the trimmed sequence reads may be extended by about 50 bp from the new start coordinate. In another embodiment, the trimmed sequence reads may be extended by about 60 bp from the new start coordinate.

As shown herein, the nucleosome regions of expressed genes are underrepresented in cfDNA, as these regions are not protected by the nucleosome and thus rapidly digested by plasma nucleases. In particular, highly expressed genes show a distinct drop in coverage in the region ˜150 bp upstream and ˜50 bp downstream of the TSS, encompassing the nucleosome depleted region (NDR).

The terms “transcriptional start site” or “TSS” as used herein refer to the location where transcription starts to the 5′-end of a gene sequence.

Methods for the identification of the TSS would be known to persons skilled in the art, illustrative examples of which include the use of an annotated reference genome or reference data sets of TSS genomic coordinates, such as refTSS (Abugessaisa et al., 2019, Journal of Molecular Biology, 431 (13): 2407-2422) and Genomic Features (Lawrence et al., 2013, PLOS Computational Biology, 9: e1003118).

The terms “nucleosome depleted region” or “NDR” as used herein refers to the genomic region encompassing the 50 bp immediately upstream of the TSS and 150 bp immediately downstream of the TSS (TSS +50 bp/−150 bp).

Nucleosome regions also comprises regulatory sequences typically associated with the promoter regions of genes including a 5′ non-coding region, a cis-regulatory region such as a functional binding site for transcriptional regulatory protein or translational regulatory protein, an upstream open reading frame, ribosomal-binding sequences, translational start site, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

The term “flanking region” as used herein refers to a genomic region encompassing the 5000 bp immediately upstream of the centre of the NDR and 5000 bp immediately downstream of the centre of the NDR (NDR +5000 bp/−5000 bp). Accordingly, the “flanking region read depth” is the number of unique sequence reads that align with the flanking region, wherein the flanking region comprises the centre of the NDR for each TSS +/−5000 bp.

In accordance with the methods disclosed herein, a genomic region comprising a TSS will be specific for a particular gene. Therefore, a person skilled in the art would appreciate that a genomic region comprising the TSS is representative of a gene.

As used herein, the term “gene” includes a nucleic acid molecule encoding an mRNA. Genes may or may not be capable of producing a functional protein. Genes can include both coding and non-coding regions (e.g., introns, regulatory elements, promoters, enhancers, termination sequences and 5′ and 3′ untranslated regions).

In accordance with the methods disclosed herein, cfDNA may be used in methods to determine gene expression from a plurality of sequence reads generated by sequencing the fragments of cfDNA.

In an embodiment, the plurality of sequence reads are generated by whole-genome sequencing (WGS). Methods for WGS would be known to persons skilled in the art and typically include amplification and sequencing of a DNA sample, in accordance with, for example, the following steps:
a. forming a nucleic acid template comprising nucleic acid(s) to be amplified or sequenced;
b. mixing the nucleic acid template(s) with one or more primers, which can hybridise to the sequence;
c. performing one or more nucleic acid amplification reactions, so that nucleic acid colonies are generated; and
d. performing sequence analysis of the nucleic colonies generated.

In an embodiment, the sequence reads are paired end sequence reads. In accordance with this embodiment, each pair of sequence reads correspond to a single cfDNA fragment.

In an embodiment, the sequence reads are at least 50 bp in length (e.g., 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 bp in length, etc.).

In an embodiment, the sequence reads are from about 50 bp to about 1000 bp in length, preferably about 50, preferably about 60, preferably about 70, preferably about 80, preferably about 90, preferably about 100, preferably about 110, preferably about 120, preferably about 130, preferably about 140, preferably about 150, preferably about 160, preferably about 170, preferably about 180, preferably about 190, preferably about 200, preferably about 210, preferably about 220, preferably about 230, preferably about 240, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, preferably about 600, preferably about 610, preferably about 620, preferably about 630, preferably about 640, preferably about 650, preferably about 660, preferably about 670, preferably about 680, preferably about 690, preferably about 700, preferably about 710, preferably about 720, preferably about 730, preferably about 740, preferably about 750, preferably about 760, preferably about 770, preferably about 780, preferably about 790, preferably about 800, preferably about 810, preferably about 820, preferably about 830, preferably about 840, preferably about 850, preferably about 860, preferably about 870, preferably about 880, preferably about 890, preferably about 900, preferably about 910, preferably about 920, preferably about 930, preferably about 940, preferably about 950, preferably about 960, preferably about 970, preferably about 980, preferably about 990, or more preferably about 1000 bp in length.

In an embodiment, the sequence reads are from about 80 bp to about 550 bp in length.

In an embodiment, the plurality of sequence reads are generated by low coverage whole-genome sequencing (LC-WGS).

LC-WGS is a next-generation sequencing method comprising amplification and sequencing of genomic regions of interest at a level of between about 0.5× to about 20× sequencing coverage. Methods for performing LC-WGS would be known to persons skilled in the art, illustrative examples of which are described by Wong et al. (2015, Cancer Research, 75 (24): 5228-5234) and Yeh et al. (2017, supra).

In an embodiment, the LC-WGS results in between about 0.5× to about 20× sequencing coverage. In another embodiment, the LC-WGS results in between about 0.5× to about 10× sequencing coverage. In an exemplary embodiment, the LC-WGS results in between 1.25× to about 10× sequencing coverage.

Analysis of the plurality of sequence reads as described herein includes performing an alignment process that determines where particular sequence reads map to a reference genome. The alignment of sequence reads may be performed using any suitable algorithm, implementation or tools known in the art, such as BWT-based (Bowtie, BWA) and hash-based (MAQ, Novoalign, Eland) aligners.

The reference genome may be the reference genome for a type of organism. In an embodiment, the reference genome is any current or future human reference genome (e.g., human reference genome assembly GRCh38, GRCh37, GRCh36, GRCh35, GRCh34, GRCh33, GRCh32, GRCh31, GRCh30, GRCh29, GRCh28, GRCh27, GRCh26, GRCh25, GRCh24, GRCh23, GRCh22, GRCh21, GRCh20, GRCh19, GRCh18, GRCh17, GRCh16, GRCh15, GRCh14, GRCh13, GRCh12, GRCh11, GRCh10, GRCh9, GRCh8, GRCh7, GRCh6, GRCh5, GRCh4, GRCh3, GRCh2, or GRCh1).

In an embodiment, the reference genome is human reference genome assembly GRCh38.

Alignment of the plurality of sequence reads may allow for the identification of sets of sequence reads that map to genomic regions comprising the TSS and the flaking region.

In an embodiment, the genomic regions comprising the TSS consist of the wider TSS region comprising positions +/−1000 with respect to the TSS (TSS +/−1000 bp). The wider region is referred to as “2K_TSS” elsewhere herein.

In another embodiment, the genomic region comprises the NDR, comprising positions −150 bp to +50 bp with respect to the TSS (TSS +50 bp/−150 bp).

In an embodiment, the method comprises the identification of sets of sequencing reads that map to genomic regions comprising the NDR (TSS +50 bp/−150 bp) and the wider TSS region (TSS +/−1000 bp).

The terms “coverage”, “read depth” and “counts” may be used interchangeably herein to refer to the number of unique sequence reads that align to genomic regions comprising the TSS.

By “corrected read depth” or “normalised read depth” it is meant read depth that has been corrected for non-expression based coverage effects as described elsewhere herein. The corrected read depth reduces or eliminates the confounding effect of copy number aberrations (CNA) and technical biases, such as GC content and mappability. For example, a gene with multiple copies (i.e., more than two) can lead to increased expression, but would also cause increased read depth at the genomic region comprising the TSS, which would lead to an inference that the gene has low expression.

In an embodiment, the corrected read depth is a continuous variable.

In an embodiment, the corrected read depth is generated by converting the flanking region read depth into log counts per million (LCPM) values using the read depth and the total number of sequence reads in the sample. For example, if the flanking read depth for a flanking region (fr) of gene (i) is frᵢ and the total number of sequence reads in the sample is N, then LCPM_frᵢ=ln(frᵢ/N)×10⁶).

In an embodiment, the corrected read depth is generated based on the read depth determined in step (e), the flanking region read depth, the total number of sequence reads in a sample and an offset count for each gene.

By “offset count” it is meant the flanking region LCPM values for a gene (LCPM_frᵢ)+the log-transformed total number of sequence reads in the sample (N), i.e., LCPM_frᵢ+ln(N). The offset count per gene may be scaled across all samples of interest to ensure that the mean offset for each gene is the same as the mean log-library size. As such, the adjustment per gene i (adjᵢ)=mean offset counts across all samples. The offset count may be scaled as mean (In (total counts across samples))+frᵢ−adjᵢ.

In an embodiment, the corrected read depth is generated by calculating the counts per million (CPM) per gene (CPMᵢ) from the read depth of the sets of sequence reads that align to genomic regions comprising a TSS (e.g., the NDRᵢ or 2K_TSSᵢ). For example. the CPMᵢ within each sample may be calculated as CPMᵢ=NDRᵢ/exp (scaled offset count)×10⁶. In another example, the CPMᵢ within each sample may be calculated as CPMᵢ=2K_TSSᵢ/exp (scaled offset count)×10⁶.

In an embodiment, the corrected read depth is the counts per million per gene (CPMᵢ).

In an embodiment, the method further comprises calculating a Z score for each genomic region using the corrected read depth and a reference value.

In an embodiment, the plurality of gene expression categories is generated by ranking the Z scores from the lowest Z score to highest Z score, and generating gene expression categories that correspond to the level of gene expression for each gene based on the ranked Z scores, wherein the genes with the lowest Z scores are grouped into high gene expression categories, and the genes with the highest Z scores are grouped into a low gene expression categories.

The term “Z score” as used herein refers to a numerical measurement that describes a value's relationship to the mean of a group of values. In accordance with the methods disclosed elsewhere herein, the Z score is calculated using the corrected read depth and a reference value.

In an embodiment, the reference value is the copy number (log2R value) of the same genomic region(s) in one or more healthy subjects.

In another embodiment, the reference value is based on the read depth of one or more reference genes in the sample. As exemplified herein, the reference genes may be identified within a sample following sample normalisation. In accordance with this embodiment, reference genes are identified by ranking all genes in a sample by the coefficient of variation (CV) of the flanking region read depth, as converted into log counts per million (LCPM), relative to the total number of sequence reads in a sample. The reference genes selected based on the sample normalization method disclosed herein represent a subset of genes with the lowest CV, e.g., the 25 genes with the lowest CV represent the “reference genes” for a sample.

The phrase “gene expression categories” as used herein refers to a bin, category, or any other group of genes that are sequentially categorised by corrected read depth or Z score. The number of genes within a gene expression category will vary according to the number of gene expression categories, with the proportion of annotated genes within each gene expression category being the same. For example, when the plurality of gene expression categories consists of 10 gene expression categories, each gene expression category will contain 10% of all annotated genes. Conversely, when the plurality of gene expression categories consists of 5 gene expression categories, each gene expression category will contain 20% of all annotated genes, and so on.

In an embodiment, each gene is ranked from lowest corrected read depth or Z score to highest corrected read depth or Z score prior to the generation of a plurality of gene expression categories. In accordance with this embodiment, the term “gene expression categories” may be alternatively referred to as a rank.

As described elsewhere herein, a genomic region comprising a TSS will be specific for a particular gene. Accordingly, the person skilled in the art would appreciate that the ranking of genomic regions comprising the TSS would be equivalent to the ranking of genes associated with the particular TSS of each genomic region.

In an embodiment, the plurality of gene expression categories consists of more than 2 gene expression categories, but not more than 20 gene expression categories, preferably 3 gene expression categories, preferably 4 gene expression categories, preferably 5 gene expression categories, preferably 6 gene expression categories, preferably 7 gene expression categories, preferably 8 gene expression categories, preferably 9 gene expression categories, preferably 10 gene expression categories, preferably 11 gene expression categories, preferably 12 gene expression categories, preferably 13 gene expression categories, preferably 14 gene expression categories, preferably 15 gene expression categories, preferably 16 gene expression categories, preferably 17 gene expression categories, preferably 18 gene expression categories, preferably 19 gene expression categories, or more preferably 20 gene expression categories.

In an embodiment, the plurality of gene expression categories consists of 10 gene expression categories, wherein each gene expression category contains 10% of all genes, such that the first gene expression category contains the 10% of all genes with the lowest corrected read depth or the lowest Z score, and the tenth category contains 10% of all genes with the highest corrected read depth or the highest Z score.

In an embodiment, the method further comprises
g. repeating steps (b) to (f) with one or more additional samples obtained from the subject at a subsequent time point(s); and
h. comparing the level of gene expression for each gene determined in step (g) with the first sample to evaluate whether there has been a change in gene expression over time.

The interval between any one or more samples may be minutes, hours, days, months or years.

In an embodiment, the interval between any one or more samples is between about 1 week to about 52 weeks, preferably about 1 week, preferably about 2 weeks, preferably about 3 weeks, preferably about 4 weeks, preferably about 5 weeks, preferably about 6 weeks, preferably about 7 weeks, preferably about 8 weeks, preferably about 9 weeks, preferably about 10 weeks, preferably about 11 weeks, preferably about 12 weeks, preferably about 13 weeks, preferably about 14 weeks, preferably about 15 weeks, preferably about 16 weeks, preferably about 17 weeks, preferably about 18 weeks, preferably about 19 weeks, preferably about 20 weeks, preferably about 21 weeks, preferably about 22 weeks, preferably about 23 weeks, preferably about 24 weeks, preferably about 25 weeks, preferably about 26 weeks, preferably about 27 weeks, preferably about 28 weeks, preferably about 29 weeks, preferably about 30 weeks, preferably about 31 weeks, preferably about 32 weeks, preferably about 33 weeks, preferably about 34 weeks, preferably about 35 weeks, preferably about 36 weeks, preferably about 37 weeks, preferably about 38 weeks, preferably about 39 weeks, preferably about 40 weeks, preferably about 41 weeks, preferably about 42 weeks, preferably about 43 weeks, preferably about 44 weeks, preferably about 45 weeks, preferably about 46 weeks, preferably about 47 weeks, preferably about 48 weeks, preferably about 49 weeks, preferably about 50 weeks, preferably about 51 weeks, or more preferably about 52 weeks.

In another embodiment, the interval between any one or more samples is between about 1 month to about 12 months, preferably about 1 month, preferably about 2 months, preferably about 3 months, preferably about 4 months, preferably about 5 months, preferably about 6 months, preferably about 7 months, preferably about 8 months, preferably about 9 months, preferably about 10 months, preferably about 11 months, or more preferably about 12 months.

In another embodiment, the interval between any one or more samples is between about 1 year to about 20 years, preferably about 1 year, preferably about 2 years, preferably about 3 years, preferably about 4 years, preferably about 5 years, preferably about 6 years, preferably about 7 years, preferably about 8 years, preferably about 9 years, preferably about 10 years, preferably about 11 years, preferably about 12 years, preferably about 13 years, preferably about 14 years, preferably about 15 years, preferably about 16 years, preferably about 17 years, preferably about 18 years, preferably about 19 years, or more preferably about 20 years.

In an embodiment, the interval between one or more samples is defined by disease status. For example, the first sample may be obtained from the subject prior to the commencement of treatment and the interval between any one or more additional samples is at the time of disease progression (i.e., relapse).

Methods for Determining the Likelihood that a Subject has Cancer

In another aspect, the present disclosure provides a method for determining the likelihood that a subject has cancer, the method comprising:
a. providing a sample obtained from the subject, wherein the sample comprises fragments of cfDNA;
b. determining the level of gene expression of one or more genes in the sample in accordance with the methods disclosed herein;
c. comparing the level of gene expression determined in step (b) with a reference level of gene expression of the one or more genes; and
d. based on the comparison in step (c) determining the likelihood that the subject has cancer.

In an embodiment, the cancer is a haematological malignancy.

In another embodiment, the haematological malignancy is selected from the group consisting of MDS and AML.

In an embodiment, the cancer is a solid tumour.

In an embodiment, the solid tumour is selected from the group consisting of breast cancer, lung cancer and melanoma.

The term “reference sample” as used herein refers to a sample from a subject, or a group of subjects who do not, and have not, had cancer (also referred to herein as “healthy control sample” or “healthy controls”); or a sample from a subject, or group of subjects who have, or have had cancer, including a specific cancer type. It is to be understood, however, that the comparison step does not need to rely on a comparison with a level of expression of one or more genes in the sample to the level of the one or more genes in a reference sample. For example, the comparison may also be made to a “reference level”; that is a known or predetermined level of expression of the one or more genes that is representative of the level of expression of the one or more genes from a subject, or a population of subjects, with or without cancer.

The “reference level” may be represented as an absolute number, or as a mean value (e.g., mean+/−standard deviation), such as when the reference level is derived from (i.e., representative of) a population of individuals (e.g., a population of subjects, with or without cancer).

In an embodiment, the reference level is a level of expression of the one or more genes that is known or predetermined from a sample obtained from one or more healthy subjects.

In an embodiment, the reference level is a level of expression of the one or more genes that is known or predetermined from a sample obtained from one or more subjects having cancer (e.g., MDS, AML, breast cancer, melanoma, lung cancer, pan-cancer, etc.).

Where the reference level is a known or predetermined level of expression of the one or more genes that is representative of the level of expression of the one or more genes from a subject, or a population of subjects with cancer, a level of gene expression that is about the same as the reference level indicates that the subject is likely to have cancer. Alternatively, wherein the reference level is a known or predetermined level of expression of the one or more genes that is representative of the level of expression of the one or more genes from a subject, or a population of subject without cancer (i.e., a healthy subject), a level of gene expression that is different to the reference level (i.e., higher or lower expression) indicates that the subject is less likely to have cancer.

In an embodiment, the one or more genes are differentially expressed in cancer subjects relative to healthy subjects.

In an embodiment, the one or more genes are characteristic of cancer type.

The terms “cancer type” or “tumour type” may be used interchangeably herein to refer to the tissue of origin of ctDNA. Persons skilled in the art would appreciate that where the one or more genes is characteristic of cancer type, the methods disclosed herein may be used to identify the tissue of origin of a cancer.

In an embodiment, the one or more genes that are characteristic of a cancer-associated pathway.

A “cancer-associated pathway” refers to signalling pathways that are associated with the development and progression of cancer. Cancer-associated pathways would be known to persons skilled in the art, illustrative examples of which include the pathways that control cell-cycle progression, apoptosis, cell growth and the oncogenic signalling pathways described by Liberzon et al., 2015, Cell Systems, 1 (6): 417-425.

Where the one or more genes are characteristic of, e.g., cancer type or a cancer-associated pathway, the one or more genes may also be referred to as a gene signature.

The term “gene signature” as used herein refers to a gene or a plurality of genes having a unique pattern of gene expression that is the consequence of either changed biological progress or an altered pathogenic state (e.g., cancer).

### Methods of Treatment

In another aspect, the present disclosure provides a method for the treatment of a subject with cancer, the method comprising:
a. providing a sample obtained from the subject, wherein the sample comprises fragments of cfDNA;
b. determining the likelihood that a subject has cancer according to the method as described herein; and
c. where based on the determination in step (b) the subject has a high likelihood of having cancer, treating the subject with a treatment for said cancer.

As used herein the terms “treat”, “treating”, “treatment”, and the like refer to any and all methods that remedy, prevent, hinder, retard, ameliorate, reduce, delay or reverse the progression of cancer or one or more undesirable symptoms thereof in any way. Thus the term “treatment” and the like are to be considered in their broadest context. For example, treatment does not necessarily imply that a patient is treated until total recovery. Cancer is characterised by multiple symptoms, and thus, the treatment need not necessarily remedy, prevent, hinder, retard, ameliorate, reduce, delay or reverse all of said symptoms. Methods of the present disclosure may involve “treating” cancer in terms of reducing or ameliorating the occurrence of a highly undesirable event or symptom associated with cancer or an outcome of the progression of cancer, but may not of itself prevent the initial occurrence of the event, symptom or outcome. Accordingly, treatment includes amelioration of the symptoms of cancer or preventing or otherwise reducing the risk of developing the symptoms of cancer.

In an embodiment, the cancer is a haematological malignancy.

In an embodiment, the haematological malignancy is MDS.

The therapeutic regimen for the MDS will typically depend on factors including, but not limited to, the stage and extent of the disease and the age, weight, and general health of the subject. Suitable treatments for MDS would be known to persons skilled in the art, illustrative examples of which include supportive care with transfusions, DNA hypomethylating agents (e.g., azacitidine, decitabine), lenalidomide, allogenic stem cell transplant, targeted therapeutic agents, and combinations thereof.

In an embodiment, the treatment comprises the administration of a DNA hypomethylating agent. In another embodiment, the treatment comprises the administration of a DNA hypomethylating agent and a thrombopoiesis-stimulating agent. In an exemplary embodiment, the treatment is a combination of azacitidine and eltrombopag.

In an embodiment, the haematological malignancy is AML.

The therapeutic regimen for the AML will typically depend on factors including, but not limited to, the stage and extent of the disease and the age, weight, and general health of the subject. Suitable treatments for AML would be known to persons skilled in the art, illustrative examples of which include combination chemotherapy (e.g., cytrabine and an anthracycline such as Idarubicin), allogenic stem cell transplant, DNA hypomethylating agents (e.g., azacitidine), targeted therapeutic agents (e.g., FLT-3 inhibitor, IDH inhibitor, BH3 mimetics), other agents (e.g., bromodomain (BET) inhibitor).

In an embodiment, the treatment is a bromodomain inhibitor. In an exemplary embodiment, the bromodomain inhibitor is molibresib.

In an embodiment, the cancer is a solid tumour.

In another embodiment, the solid tumour is selected from the group consisting of breast cancer, lung cancer and melanoma.

The therapeutic regimen for a solid tumour will typically depend on factors including, but not limited to, the stage and extent of the disease, hormone receptor status and the age, weight, and general health of the subject.

Suitable treatments for breast cancer would be known to persons skilled in the art, illustrative examples of which chemotherapy (e.g., docetaxel, cyclophosphamide, adriamycin, paclitaxel), radiation therapy, hormone therapy (e.g., tamoxifen, aromatase inhibitors (e.g., anastrozole, exemestane and letrozole), targeted therapeutic agents (e.g., palbociclib, trastuzumab), other agents (e.g., bromodomain (BET) inhibitor) and other treatment modalities described by, for example, Waks and Winer, 2019, JAMA, 321 (3): 288-300.

In an embodiment, the treatment is selected from the group consisting of palbociclib, letrozole, and combinations thereof.

Suitable treatments for lung cancer would be known to persons skilled in the art, illustrative examples of which include surgery, chemotherapy (e.g., carboplatin, cisplatin, docetaxel, gemcitabine), radiation therapy, targeted therapy, immunotherapy or a combination of these treatment modalities.

In an embodiment, the treatment is an epidermal growth factor receptor tyrosine kinase inhibitor.

Suitable treatments for melanoma would be known to persons skilled in the art, illustrative examples of which include surgery, immunotherapy, targeted therapy (e.g., MAPK inhibitor therapy), chemotherapy and radiation therapy.

In an embodiment, the treatment is selected from MAPK inhibitor therapy and immunotherapy.

It is further contemplated herein that the determination of gene expression from cfDNA as described herein is useful in the stratification of patients to a particular therapeutic regimen with a higher likelihood of treatment benefit. For example, it is known in the art that despite azacitidine being the cornerstone for the clinical management of patients with high-risk MDS, only approximately half of MDS patients respond to azacitidine-based therapy (Silverman et al., 2002, Journal of Clinical Oncology, 20 (10): 2429-2440). Accordingly, it is demonstrated that therapeutic response in MDS patients may be at least partially determined by gene expression signatures that are derived from cfDNA. On this basis, it is reasonable to expect that the methods described herein may also be useful for the stratification of patients who may respond to treatments based on their determined gene expression profiles as measured from cfDNA.

### Methods for Monitoring Disease Status in a Subject Having Cancer

In another aspect, the present disclosure provides methods for monitoring disease status in a subject having cancer, the method comprising:
a. providing a first sample obtained from the subject, wherein the sample comprises fragments of cfDNA;
b. determining the level of gene expression of one or more genes in the first sample according to the method as described herein;
c. repeating step (b) with one or more additional samples obtained from the subject at a subsequent time point(s);
d. determining the tumour fraction (%) for the first sample and the one or more additional samples;
e. normalising the level of gene expression in each sample determined in steps (b) and (c) based on tumour fraction; and
f. comparing the normalised level of gene expression for each gene in the first sample with the normalised level of gene expression for each gene in the one or more additional samples to evaluate whether there has been a change in gene expression over time.

The term “tumour fraction” as used herein refers to the fractional proportion of tumour DNA relative to total cfDNA. Tumour fraction is dependent on multiple factors, e.g., disease extent (localized vs metastatic), overall tumour burden, disease status (e.g., progressing, stable or responding to treatment), patient-context factors (e.g., fasting status or physical activity prior to blood collection), and technical factors (e.g., sample acquisition, transport and sample processing procedures).

In an embodiment, the determined gene expression (e.g., CPMᵢ) of the one or more genes is normalised based on the tumour fraction of each sample, wherein the cfDNA fraction (i.e., the covariate) is defined as 1 minus the tumour fraction of each sample.

In an embodiment, the first sample is a baseline sample obtained from the subject prior to the commencement for a treatment for the cancer.

In an embodiment, the additional samples are obtained from the subject at a subsequent time point selected from the group consisting of during treatment, after treatment and both during and after treatment.

In an embodiment, the disease status of the subject is selected from responsive to the treatment and resistant to the treatment.

In an embodiment, the disease status of the subject is selected from remission and relapse.

In an embodiment, the cancer is a haematological malignancy.

In an embodiment, the haematological malignancy is MDS.

Suitable treatments for MDS would be known to persons skilled in the art, illustrative examples of which include supportive care with transfusions, DNA hypomethylating agents (e.g., azacitidine, decitabine), lenalidomide, allogenic stem cell transplant, targeted therapeutic agents, and combinations thereof.

In an embodiment, the treatment comprises the administration of a DNA hypomethylating agent. In another embodiment, the treatment comprises the administration of a DNA hypomethylating agent and a thrombopoiesis-stimulating agent. In an exemplary embodiment, the treatment is a combination of azacitidine and eltrombopag.

In an embodiment, the haematological malignancy is AML.

Suitable treatments for AML would be known to persons skilled in the art, illustrative examples of which include combination chemotherapy (e.g., cytrabine and an anthracycline such as idarubicin), allogenic stem cell transplant, DNA hypomethylating agents (e.g., azacitidine), targeted therapeutic agents (e.g., FLT-3 inhibitor, IDH inhibitor, BH3 mimetics), other agents (e.g., bromodomain (BET) inhibitor).

In an embodiment, the treatment is a bromodomain inhibitor. In an embodiment, the bromodomain inhibitor is molibresib.

In an embodiment, the cancer is a solid tumour.

In another embodiment, the solid tumour is selected from the group consisting of breast cancer, lung cancer and melanoma.

Suitable treatments for breast cancer would be known to persons skilled in the art, illustrative examples of which include chemotherapy (e.g., docetaxel, cyclophosphamide, adriamycin, paclitaxel), radiation therapy, hormone therapy (e.g., tamoxifen, aromatase inhibitors (e.g., anastrozole, exemestane and letrozole), targeted therapeutic agents (e.g., palbociclib, trastuzumab), other agents (e.g., bromodomain (BET) inhibitor) and other treatment modalities described by, for example, Waks and Winer, 2019, supra.

In an embodiment, the treatment if selected from the group consisting of palbociclib, letrozole, and combinations thereof.

Suitable treatments for lung cancer would be known to persons skilled in the art, illustrative examples of which include surgery, chemotherapy (e.g., carboplatin, cisplatin, docetaxel, gemcitabine), radiation therapy, targeted therapy, immunotherapy or a combination of these treatment modalities.

In an embodiment, the treatment is an epidermal growth factor receptor tyrosine kinase inhibitor.

Suitable treatments for melanoma would be known to persons skilled in the art, illustrative examples of which include surgery, immunotherapy, targeted therapy, chemotherapy and radiation therapy.

In an embodiment, the treatment is selected from a MAPK inhibitor and immunotherapy.

It is further contemplated herein that monitoring disease status using plasma cfDNA as described herein is useful in monitoring for non-genomic transcriptional evolution or adaptation, with a view to detecting treatment resistance, trans-differentiation or linage switching to inform more effective treatment strategies.

The interval between any one or more samples may be minutes, hours, days, months or years.

In an embodiment, the interval between any one or more samples is between about 1 week to about 52 weeks, preferably about 1 week, preferably about 2 weeks, preferably about 3 weeks, preferably about 4 weeks, preferably about 5 weeks, preferably about 6 weeks, preferably about 7 weeks, preferably about 8 weeks, preferably about 9 weeks, preferably about 10 weeks, preferably about 11 weeks, preferably about 12 weeks, preferably about 13 weeks, preferably about 14 weeks, preferably about 15 weeks, preferably about 16 weeks, preferably about 17 weeks, preferably about 18 weeks, preferably about 19 weeks, preferably about 20 weeks, preferably about 21 weeks, preferably about 22 weeks, preferably about 23 weeks, preferably about 24 weeks, preferably about 25 weeks, preferably about 26 weeks, preferably about 27 weeks, preferably about 28 weeks, preferably about 29 weeks, preferably about 30 weeks, preferably about 31 weeks, preferably about 32 weeks, preferably about 33 weeks, preferably about 34 weeks, preferably about 35 weeks, preferably about 36 weeks, preferably about 37 weeks, preferably about 38 weeks, preferably about 39 weeks, preferably about 40 weeks, preferably about 41 weeks, preferably about 42 weeks, preferably about 43 weeks, preferably about 44 weeks, preferably about 45 weeks, preferably about 46 weeks, preferably about 47 weeks, preferably about 48 weeks, preferably about 49 weeks, preferably about 50 weeks, preferably about 51 weeks, or more preferably about 52 weeks.

In another embodiment, the interval between any one or more samples is between about 1 month to about 12 months, preferably about 1 month, preferably about 2 months, preferably about 3 months, preferably about 4 months, preferably about 5 months, preferably about 6 months, preferably about 7 months, preferably about 8 months, preferably about 9 months, preferably about 10 months, preferably about 11 months, or more preferably about 12 months.

In another embodiment, the interval between any one or more samples is between about 1 year to about 20 years, preferably about 1 year, preferably about 2 years, preferably about 3 years, preferably about 4 years, preferably about 5 years, preferably about 6 years, preferably about 7 years, preferably about 8 years, preferably about 9 years, preferably about 10 years, preferably about 11 years, preferably about 12 years, preferably about 13 years, preferably about 14 years, preferably about 15 years, preferably about 16 years, preferably about 17 years, preferably about 18 years, preferably about 19 years, or more preferably about 20 years.

In an embodiment, the interval between one or more samples is defined by disease status. For example, the first sample may be obtained from the subject prior to the commencement of treatment (i.e., the baseline sample) and additional sample is obtained at the time of disease progression (i.e., relapse).

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### General Methods

### Patients

The cohort of 12 MDS patients described herein were enrolled in a Phase I clinical study assessing azacitidine and eltrombopag for the treatment of high-grade MDS. The study was approved by the Peter MacCallum Cancer Centre research ethics committee (10/78 and 17/56).

The 2 AML patients described herein were enrolled in a Phase I clinical study assessing the BET inhibitor GSK525762 (molibresib) for the treatment of AML. The study was approved by the Peter MacCallum Cancer Centre research ethics committee (14/105 and 17/56).

The cohort of 41 breast cancer patients described herein were separated into 2 cohorts. Cohort 1 included 7 breast cancer patients recruited as part of the Metastatic Breast Circulating Biomarker (M-BCB) protocol at the Peter MacCallum Cancer Centre. These 7 patients all underwent treatment with a CDK4/6 inhibitor, palbociclib, in combination with the aromatase inhibitor, letrozole.

Cohort 2 included a separate subset of patients with metastatic breast cancer, with various breast cancer phenotypes and treatment received. The study was approved by the Peter MacCallum Cancer Centre research ethics committee (15/72).

The lung cancer cohort comprised 10 patients with stage IV non-small cell lung cancer (EGFR mutation positive). These patients underwent treatment with epidermal growth factor tyrosine kinase inhibitor (osimertinib). The study was approved by the Peter MacCallum Cancer Centre research ethics committee (11/88).

The melanoma cohort comprised 40 patients with stage IV melanoma (BRAF mutation positive). These patients underwent treatment with a MAPK inhibitor therapy or immunotherapy. A subset of these patients (n=3) underwent treatment with dabrafenib and trametinib. The study was approved by the Peter MacCallum Cancer Centre research ethics committee (11/105).

Written informed consent to participate in these studies was provided by all patients.

### Plasma DNA Sample Preparation

Whole blood was collected in Acid Citrate Dextrose (ACD) anti-coagulant tubes (Sarstedt S-Monovettes, 8.5 mL CPDA) or in Ethylenediaminetetraacetic acid (EDTA) anti-coagulant tubes (Interpath, 9 ml VACUETTE K3) and processed within four hours of collection.

Whole blood was first centrifuged at 1600 g for 10 minutes (brake off) to separate the plasma from the peripheral blood cells, followed by a further centrifugation step at 20,000 g for 10 minutes to pellet any remaining cells and/or debris. The plasma was then stored at −80° C. prior to extraction of DNA.

DNA was extracted from aliquots of plasma using the QIAamp Circulating Nucleic Acid Kit (Qiagen, #55114) according to the manufacturer's instructions. Estimation of the efficiency of DNA extraction was performed as previously described by Dawson et al. (2013, New England Journal of Medicine, 368 (13): 1199-1209) using PCR amplified genomic DNA amplicon from Drosophila melanogaster.

### Low Coverage Whole-Genome Sequencing (LC-WGS)

Libraries for low coverage whole genome sequencing were prepared using the NEBNext® Ultra™ II DNA Library Prep Kit (Illumina®) according to the manufacturer's instructions.

Sequencing was performed on either the Illumina Nextseq (paired-end 75 bp reads) or the Illumina Novaseq platform (paired-end 100 and 150 bp reads) according to the manufacturer's instructions.

Sequencing reads were aligned to the human genome (build hg38) using bwa-mem (version 0.7.13) with alternate-contig aware mapping. Presumed PCR and optical duplicates were marked using Picard tools software suite (version 2.17.3) and subsequently filtered along with any alignments not considered as primary alignments or having a mapping quality less than a Phred score of 30. Samtools (version 1.9) 34 was used to down-sample reads to either simulate decreasing coverage depths and equalise the starting coverage of samples. Unless otherwise stated, all analyses have been carried out on LCWGS with read counts capped at ˜ 170K such that the genome-wide mean coverage per sample was approximately 5.5× for 100 bp reads.

### RNA Sample Preparation and Full-Length RNA Sequencing

Matched bone marrow (BM) aspirate samples taken from MDS patients were processed by the Ficoll-Pacque Plus (GE Healthcare, #17144002) separation method according to the manufacturer's instructions to obtain BM-mononuclear leukocytes (BM-MNL). BM-MNL RNA was extracted using the RNeasy Mini Kit (Qiagen, #74104 and 74106) according to manufacturer's instructions. Whole bone marrow RNA was extracted using TRIzol Reagent (Invitrogen™, #15596026 and 15596018) according to manufacturer's instructions.

Libraries for full-length RNA sequencing were prepared using the NEBNext® Ultra™ II Directional RNA Library Prep Kit for Illumina® and NEBNext Poly (A) mRNA Magnetic Isolation Module were used for library preparation according to the manufacturer's instructions.

Sequencing was performed on either the Illumina Nextseq or the Illumina Novaseq platform according to the manufacturer's instructions.

RNA-seq reads were aligned to the hg38 human reference genome using Histat2 software (version 2.1.0; Kim et al., 2015, Nature Methods, 12:357-360) and read counts per gene were determined using “featureCounts” from the Rsubread package, filtering for multi-mapping reads and accounting for their paired nature. Raw gene counts were transformed into counts per million (CPM), accounting for library size differences with the trimmed mean of M-values (TMM) normalization within the edgeR package (McCarthy et al., 2012, Nucleic Acids Research, 40:4288-4297). The generalized linear model-based approach implemented in edgeR was used to assess differential gene expression between the sample groups of interest. A gene was considered as significantly differentially expressed when its false discovery rate-adjusted P value was <0.05. Heatmaps of the genes with the largest absolute log fold changes were generated using the pheatmap R package.

RNA-seq reads were also aligned to the GRCh38 human reference genome using the subread software (version 1.5.2; Liao et al., 2013, Nucleic Acids Research, 41: e108). Read counts at the gene level were determined using function featureCounts in the Rsubread package by summing across technical replicates, accounting for the paired nature of reads and in a strand specific manner. Raw gene counts were transformed into fragments per kilobase per million reads (FPKM) to assess the gene-wise expression levels for within-sample analyses. Differentially expressed genes were assessed using a negative binomial generalized log-linear model using the edgeR package (McCarthy et al., 2012, supra). A gene was considered as significantly differentially expressed when its false discovery rate-adjusted P value was <0.05.

Single-Cell RNA (scRNA) Analysis

Single cell RNA sequencing data for AML patients BET001 and BET002 from flow selected blasts in BM-MNL at baseline, remission and relapse time points was provided by Bell et al. (2019, supra).

### Analysis Methods

### Alignment of LC-WGS Paired-End Reads

Paired-end sequence reads were aligned to human reference genome assembly GRCh38 (hg38) using bwa mem, version 0.7.13 (Li & Durbin, 2009, Bioinformatics, 25 (14): 1754-1760) with alternate-contig aware mapping. This alignment provides the exact genomic coordinates of the start and end of each read. Presumed PCT and optical duplicates were marked using Picard tools software suite (version 2.17.3) and subsequently filtered along with any alignments not considered as primary alignments or having a mapping quality less than a Phred score of 30. Samtools (version 1.9) was used to down sample reads in all samples such that the genome-wide mean coverage per sample was approximately 5×.

Blacklisted regions defined by Amemiya et al., 2019, Scientific Reports, 9:9354 as curated from ENCODE (i.e., ENCODE Data Analysis Centre (DAC) blacklisted regions) were filtered from all samples.

### Copy Number Analysis of LC-WGS

Somatic copy number alteration (CNA) analysis was performed using the QDNAseq pipeline (version 1.26.0; Scheinin et al., 2014, Genome Research, 24:2022-2032) modified for GRCh38. First, the genome was divided into non-overlapping 50 Kbp bins. The number of reads overlapping each bin was counted and adjusted with a simultaneous two-dimensional loess correction accounting for read mappability and GC content in each region and the interaction of these two factors. Genomic bins with possible count artefacts were filtered using a blacklist generated from the healthy control cohort and the 1000 Genomes project (Auton et al., 2015, Nature, 526:68-74). Read counts in each bin were then normalized by the median genome-wide count of each sample to obtain relative copy number estimates in the form of log₂ ratios. Finally, bins were segmented using the Circular Binary Segmentation algorithm, to merge regions of similar copy number together.

These final CNA profiles were used to investigate possible ‘genomic’ evolution in serial plasma samples within patients. The segmented copy number values were also used within the accuracy analysis as an input to the Ulz et al., (2016, supra) algorithm to correct for changes in coverage around transcription start sites (TSS) due to copy number alterations.

Inferring Tumor Fraction from LC-WGS

Tumor fraction was estimated in each sample using ichorCNA (version 0.3.2; Adalsteinsson et al., 2017, Nature Communications, 8:1324) using a window size of 1 Mb (-window), restricting counts to autosomes (-chromosome) and optimising other settings for low ctDNA fractions in plasma. Namely, we initialised the non-tumour fractions to 0.95, 0.99, 0.995 and 0.999 (-normal), initialised the ploidy of samples to 2 (-ploidy), set a maximum copy number of 3 (-maxCN) and turned off the subclonal CNA detection mode. Furthermore, a panel of normal was constructed from the healthy control cohort to normalise data to reduce noise and improve accuracy.

### Obtaining Fragment Counts at Transcription Start Sites (TSS)

Gene transcription start sites (TSS) for hg38 human reference were downloaded from UCSC Genome Browser using the “NCBI RefSeq Select” option. This dataset consists of a representative or “Select” transcript for every protein-coding gene curated using specific criteria listed in www.ncbi.nlm.nih.gov/refseq/refseq_select/. For each TSS, the nucleosome depleted region (NDR) was defined as the positions from −150 bp to +50 bp with respect to the TSS.

For each sample, sequenced fragments that overlap with the NDR sites were counted. In accordance with this method, a single base overlap was counted towards the NDR coverage (i.e., flanking region read depth).

### Correcting for Non-Gene Expression-Based Coverage Effects

Copy number aberrations (CNA) and technical biases such as GC content and mappability can influence the counts at each NDR. In this context, where CNA is present, it has a major confounding effect. For example, a gene with multiple copies can lead to increased expression, but would also result in an increased read depth, which may lead to an inference that the gene has low expression in accordance with the methods described herein. Accordingly, to normalise for these confounding effects, the following protocol was implemented:
1. The number of fragments falling within 5000 bp on either side of the centre of each NDR were counted (i.e., flanking region read depth);
2. The flanking region read depth was then converted to log counts per million (LCPM) values, i.e., if the flanking region count of a gene i is frᵢ and the total number of fragments in the sample is N, then LCPM fr of gene i=ln((frᵢ/N)×10⁶);
3. An “offset count” was formulated for each gene i by adding the log-transformed library size (N) to the LCPM calculated at step 2, i.e., LCPM fr+ln(N);
4. The offset count per gene were scaled across all samples of interest ensuring that the mean offset count for each gene is the same as the mean log-transformed library size, for example, the adjustment for each gene (e.g., gene i) may be described as follows:
   Adjustment for gene i (adjᵢ)=mean value of offset counts across all samples
   Scaled offset count for gene i in a given sample=mean (ln(total counts across samples))+frᵢ-adjᵢ
5. The read depth for each genomic region comprising the TSS was normalized for each NDR (or for the 2K_TSS region) within each sample, for example,

$$C P {M}_{i} = N D {R}_{i} count / exp ( scaled offset count ) * {10}^{6}$$

Steps 4 and 5 of the correction protocol are performed using the calculateCPM function in the csaw R package.

### Sample Normalisation

Samples were normalised to ensure that the percentile rank for the flanking region read count for each gene has a CPM of <0. As there is a continuous distribution of between 0 to 1, genes with low expression (i.e., high CPM counts) will have a percentile closer to 1.

For each gene, the mean and SD of its percentile across all samples was calculated. Thereafter, a coefficient of variation (CV) was calculated per gene, which is defined as its SD divided by the mean.

25 reference genes were then selected based on the smallest CV values. These genes would be expected to have high counts but also have low variability across samples.

### Z Score

Z scores were calculated for each gene per sample by calculating mean and standard deviation (SD) for the 25 reference genes using the flanking region read depth. By subtracting the reference mean from the corrected read count for each gene, then dividing by the reference gene SD (i.e., reference value), Z scores relative to reference gene flanking region read depth was calculated for each gene per sample.

### Clustering Analysis

Unsupervised clustering was performed using the Z scores of all genes. For the purpose of this analysis, genes were omitted if a single sample has a CPM value of 0, or if the gene has a Z-score greater than 0, which would indicate that the gene is less expressed than the reference genes.

The remaining genes were dimensionality reduced via a Principal Component Analysis, with the samples plotted according to the coordinates in either PC1, PC2 or PC3.

When discriminating between cancer samples and healthy controls, receiver operating characteristic (ROC) curves were plotted by defining a score using the PCA coordinates (e.g., PCA score—PC2 coordinate+PC3 coordinate), with this score then used as a discriminatory variable to classify cancer samples from healthy controls.

Non-Invasive Plasma Expression Ranking Algorithm with Z-Score Categorisation

Sequencing reads from LC-WGS of plasma were aligned to the human genome and the mapping locations of the two extreme ends of each read pair were used to estimate the approximate size of each DNA fragment sequenced. To enhance the nucleosome-specific coverage signal, the mapped DNA fragment was trimmed to remove parts of the alignment more likely to be associated with the DNA linker regions. Fragments shorter than 80 bp were discarded as well as reads that were improperly paired. For fragments between 80 bp-169 bp in length, the start coordinate of the DNA fragment was moved to: new start=original start position+(fragment length−60 bp)/2. For fragments between 170-250 bp in length (i.e., fragments most likely to contain 1 nucleosome and part of the linker DNA connecting to the next nucleosome), the start coordinate was moved to: new start=original start+(fragment length−60 bp)/3. For fragments of length >250 bp (fragments most likely to encompass at least 2 nucleosomes along with linker DNA adjoining them): new start=original start+(fragment length−60 bp)/4. All fragments were then extended 60 bp from the new start positions.

The coverage value contributed by the trimmed fragments at and around each RefSeq annotated TSS were extracted using the samtools depth function. For each TSS, the nucleosome depleted region (NDR) was defined as the positions from −150 bp to +50 bp with respect to the TSS. For each sample the number of sequence fragments that overlap with the NDR sites were counted, a single base overlap with the NDR sites was sufficient to be counted towards the coverage value (i.e., read depth).

The read depth was then corrected to generate a corrected read depth, which removed any confounding effects associated with copy number aberrations (CNA) and other technical biases such as GC content and mappability.

Finally, these corrected read depth values were integrated into a Z score for each gene per sample by calculating mean and standard deviation (SD) for 25 reference genes using the flanking region read depth. Z scores for each gene were then calculated by subtracting the reference mean from the corrected read count for each gene, then dividing by the reference gene SD (i.e., reference value). Accordingly, the Z scores calculated according to the methods disclosed herein are relative to the reference gene flanking region read depth.

The distribution of these integrated scores across all annotated genes was divided into 10 ranks using the decile estimates in each sample. The optimal number of ranks to subdivide the coverage score distribution depends on the depth of sequencing and the proportion of tumour DNA present in plasma. Ten ranks of plasma expression provided statistically significant discriminatory power when compared directly to expression values in RNA-seq. On account of the reciprocal nature of TSS coverage and gene expression level in plasma, genes annotated as rank 1 (with the lowest Z scores) were assumed to have the highest expression level with rank 10 genes assumed to have the lowest expression. Genes with a coverage score of zero were filtered prior to downstream analyses as this was more likely to be an effect of no reads mapping to these regions on account of low sequencing depth, rather than due to true biological variation.

Quantitative Analysis to Compare RNA-Seq Expression with Plasma-Based Expression

To test the concordance of the plasma-based gene expression values with matched RNA-seq data, every TSS was annotated from each MDS sample with the TMM-normalized fragment per kilobase million (FPKM) values of the respective genes generated from the bone marrow RNA-seq analysis. The FPKM values were sorted to identify the most-highly and least-expressed genes.

Similarly, the FPKM values that fell under each expression rank as annotated using plasma coverage, were compared in pair-wise Wilcoxon rank-sum tests to determine if they were statistically significant different distributions. For genes that had multiple TSS annotations, the plasma-based gene expression ranks were averaged in order to compare with the matched FPKM value for the gene from RNA-seq.

### Serial Non-Invasive Plasma Expression Ranking Algorithm (SNIPER)

Sequencing reads from LC-WGS of plasma were aligned to the human genome and the mapping locations of the two extreme ends of each read pair were used to estimate the approximate size of each DNA fragment sequenced. To enhance the nucleosome-specific coverage signal, the mapped DNA fragment was trimmed to remove parts of the alignment more likely to be associated with the DNA linker regions. Fragments shorter than 80 bp or longer than 550 bp were discarded, as well as reads that were improperly paired. For fragments between 80 bp-169 bp in length, the start coordinate of the DNA fragment was moved to: new start=original start position+(fragment length−50 bp)/2. For fragments between 170-250 bp in length (i.e., fragments most likely to contain 1 nucleosome and part of the linker DNA connecting to the next nucleosome), the start coordinate was moved to: new start=original start+(fragment length−50 bp)/2.5. For fragments of length between 250 bp-350 bp (fragments most likely to encompass at least 2 nucleosomes along with linker DNA adjoining them), the start coordinate was moved to: new start=original start+(fragment length−50 bp)/3. For fragments of a length between 350 bp-550 bp (i.e., fragments most likely to encompass at least 3 nucleosomes along with the linker DNA adjoining them), the start coordinate was moved to: new start=original start+(fragment length−50 bp)/4. All fragments were then extended 50 bp from the new start positions.

The coverage value (i.e., read depth) contributed by the trimmed fragments at and around each RefSeq annotated TSS were extracted using the samtools depth function. For each TSS, the NDR was defined as the positions from −150 bp to +50 bp with respect to the TSS. A larger region, termed 2K_TSS, was defined as positions +/−1000 bp on either side of the TSS. For each sample the number of trimmed sequence fragments that overlap with one or both of the NDR and 2K_TSS sites were counted, a single base overlap with the NDR sites was sufficient to be counted towards the coverage value (i.e., “NDR counts” or “2K_TSS counts”).

The NDR counts or 2K_TSS counts were then normalised to generate a corrected read depth (e.g., CPMᵢ), which removed any confounding effects associated with copy number aberrations (CNA) and other technical biases such as GC content and mappability, as detailed above at paragraph [0198].

Accuracy Estimation and Concordance Analysis with Matched RNA-Seq Data

Accuracy was estimated within a support vector machine model (SVM) framework to compare the method described herein with that of Ulz et al., (2016, supra). Training genes were identified from TCGA RNA-seq data from the UCSC Toil RNAseq Recompute Compendium (Vivian et al., 2017, Nature Biotechnology, 35:314-316). Gene-wise coefficients of variation were used to select 1000 genes that either had consistently high or low gene expression across all samples from 20 tumour types using gene-wise coefficient of variation as the training dataset.

A subset of 300 expressed genes and 300 unexpressed genes were randomly selected from the training dataset and their CPMᵢ values were used to train a SVM model for each baseline sample in the MDS cohort. The model was used to classify all other genes not included in the N=600 training gene set. This was repeated 1,000 times, each time selecting a different subset of training genes. A gene was considered to be expressed when the prediction consent of all the iterations was higher than 75%. Another SVM model was built on the same training data using CPMᵢ values from both the NDR and 2K_TSS regions per gene. A third SVM model was built on the same training data using CPMᵢ values from the NDR and 2K_TSS regions per gene, in order to compare the method described herein with that of Ulz et al., (2016, supra), which uses a two feature classifier.

To test the accuracy of the method, we identified the top 500/1000 and 5000 most-highly and least-expressed genes from the matched RNA-seq dataset for each sample. The accuracy estimates of the SVM model-based predictions were calculated using the categories in Table 1.

TABLE 1
Accuracy estimates of SVM model-based predictions

| | | Plasma-based classifications | |
|---|---|---|---|
| | | Expressed | Non-expressed |
| RNA-seq | Top 500 genes | True positives | False negatives |
| | Bottom 500 genes | False positives | True negatives |

Differential Analysis of Plasma cfDNA-Derived Gene Expression

Differentially expressed genes between serial time points were assessed using the limma empirical Bayes method (Smyth et al., 2004, Statistical Applications in Genetics and Molecular Biology, 3:1-25). The CPMᵢ values at the NDRs were modelled by both time point (categorical variable) and cfDNA fraction (covariate). Here, the cfDNA fraction was defined as 1 minus the tumor fraction of each sample as obtained from ichorCNA.

### Pathway Enrichment Analysis

Query gene lists curated from differential expression analysis using the plasma cfDNA method or RNA-seq data, were assessed for enriched biological pathways using the gprofiler2 package (version 0.1.8; Reimand et al., 2007, Nucleic Acids Research, 35: W193-200; Reimand et al., 2019, Nature Protocols, 470:187-517). Genes associated with the 50 HALLMARK pathways were downloaded from the Molecular Signatures Database (version v7.4) for this purpose. The pathway enrichment analysis was carried out with the statistical background set to match the exact gene annotations (i.e., TSS annotations) used in the plasma cfDNA method and multiple testing correction defined as the Set Counts and Sizes (SCS) method, which was shown to reduce false positive findings. The analysis generated two estimates that quantified the enrichment of each pathway tested:
a. The precision score is the proportion of genes in the input gene list (query) that overlap with each pathway (defined as intersection_size/query_size); and
b. The recall score is the proportion of functionally annotated genes of each pathway (term) that the input gene list recovers (defined as intersection_size/term size). We integrated both these estimates within an enrichment metric, which is the harmonic mean of precision and recall, defined as 2×(precision×recall)/(precision+recall), in order to quantify and visualize differences in the enrichment level between HALLMARK pathways in an unbiased manner.

### Example 1—Gene Expression Determined from cfDNA Correlates with RNA Sequencing

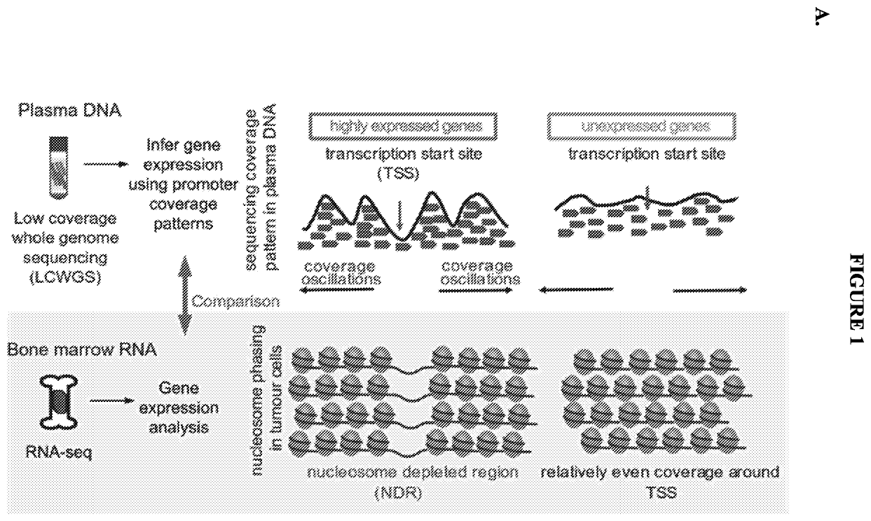

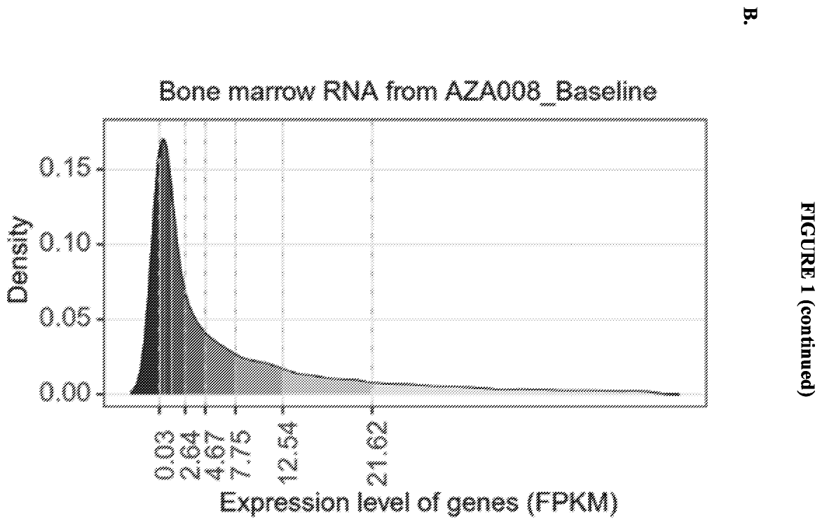

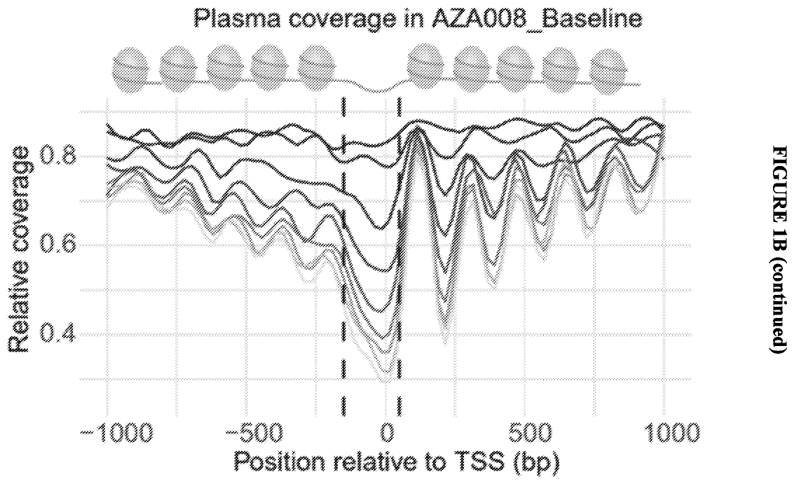

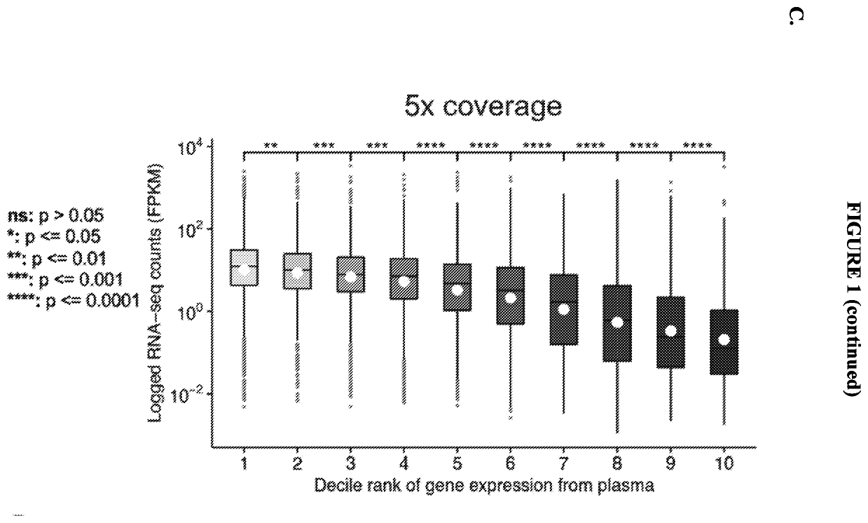

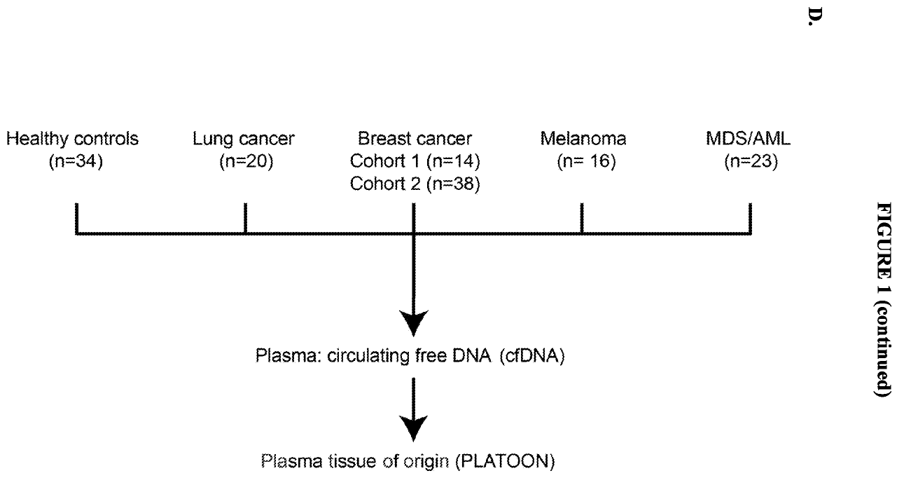

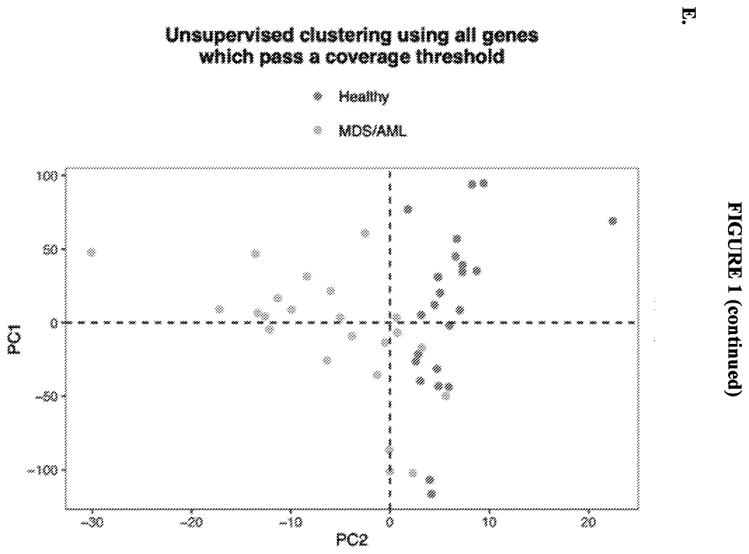

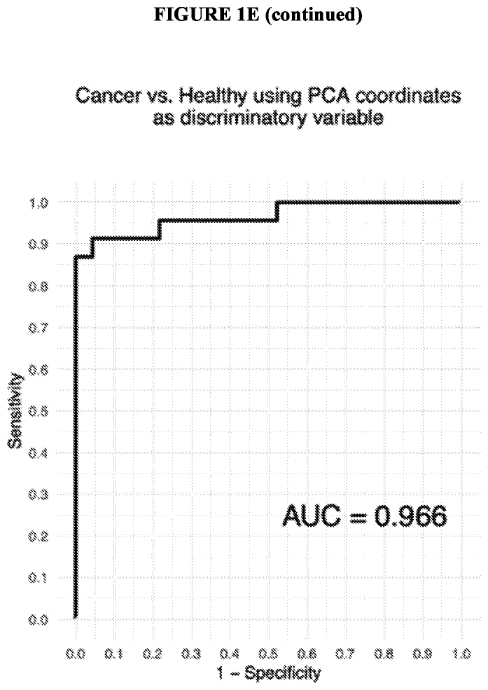

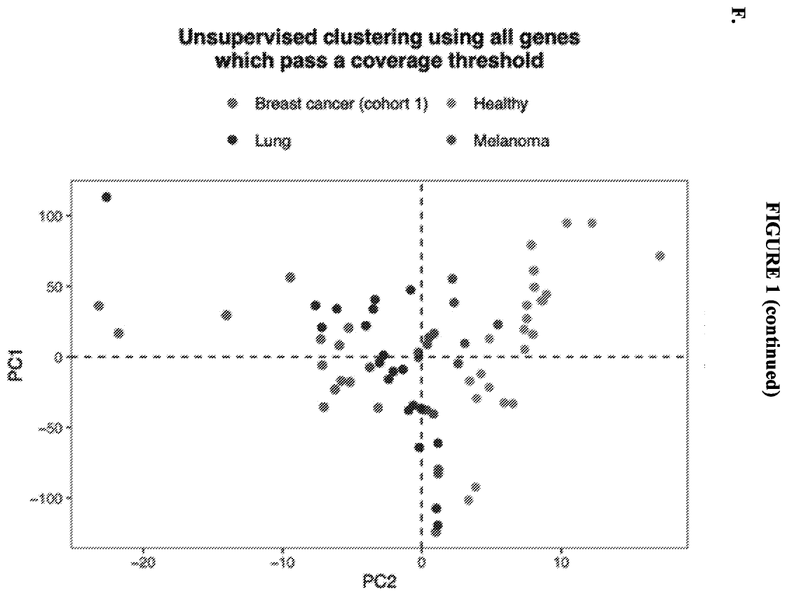

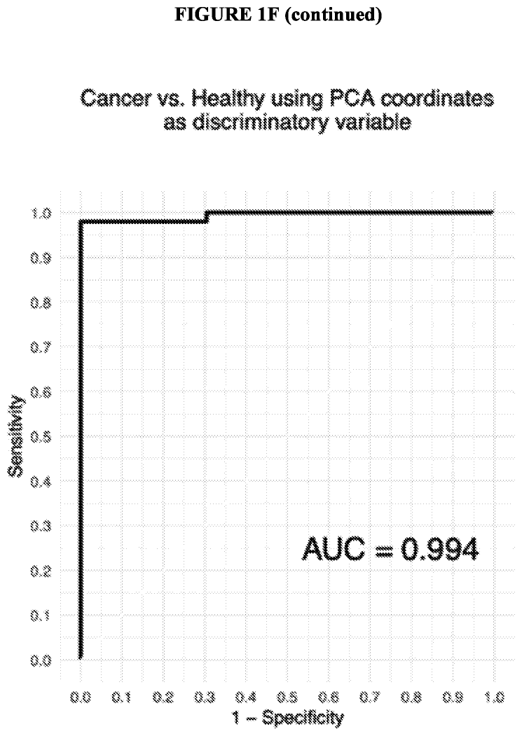

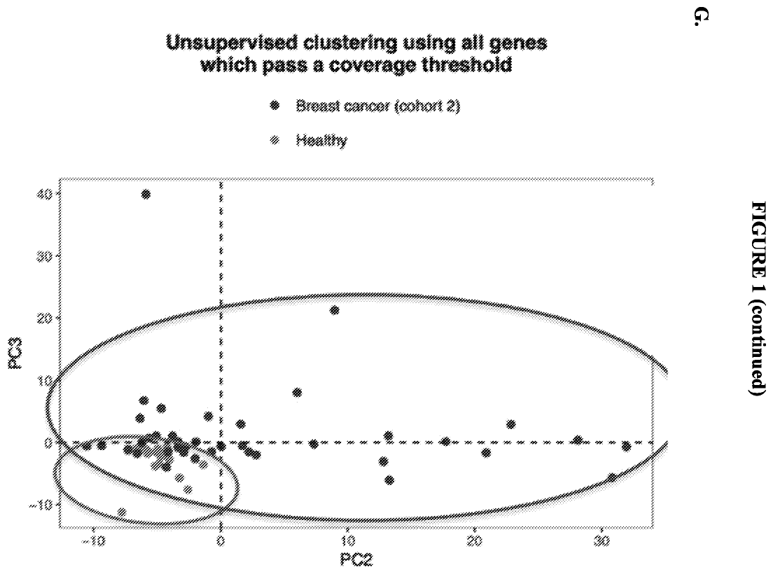

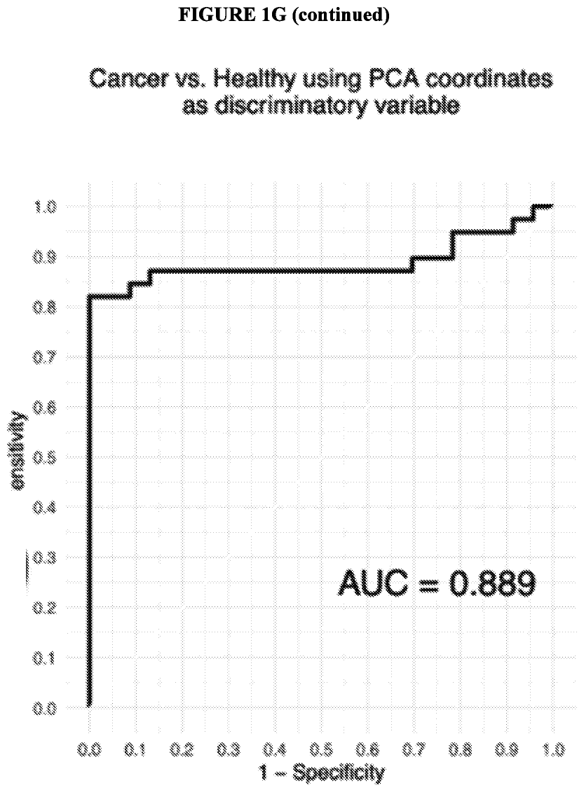

Cohorts of patients with lung cancer (n=20), breast cancer (n=14 and 38), melanoma (n=16) and a combined cohort of myelodysplastic syndrome (MDS) and acute myeloid leukaemia (AML) patients (n=23) were used in an initial study to assess the viability of using cell-free DNA (cfDNA) to determine gene expression (FIG. 1D). Low coverage whole genome sequencing (LC-WGS) was performed using the plasma samples and full-length RNA sequencing (RNA-seq) was performed on the whole bone marrow aspirate samples as a reference for tumour gene expression profiles.

In whole genome sequencing data from plasma, coverage patterns at transcriptional start sites (TSS) were observed to change with gene expression levels. In particular, there was an incremental decrease in read depth over the TSS corresponding to the level of gene expression according to bone marrow RNA-seq data, highlighting the ability of cfDNA to accurately represent and rank actively transcribed genes in the marrow of MDS patients (FIG. 1B). A comparison of the most highly expressed genes in the bone marrow compared to the gene expression determined from plasma indicated a high degree of concordance between both compartments (FIG. 1B). These data indicate that genomic features of cfDNA in plasma may be used to accurately determine gene expression with a level of accuracy and sensitivity that is similar to RNA sequencing of whole bone marrow.

Given the unexpected correlation between bone marrow and plasma gene expression, we then developed a novel gene expression rank score in plasma cfDNA. This methodology differs from previous methods of inferred gene expression (e.g., Ulz et al., 2016, supra) as it is able to accurately differentiate gene expression in plasma cfDNA into at least 10 gene expression categories, as compared to the binary value or previous methods (FIG. 1C).

Using clustering analysis of the Z scores generated for each gene, MDS and AML patients were clearly distinguished from healthy controls (FIG. 1E). The distinction between cancer cases and healthy controls was also replicated in the context of the solid tumour cohorts, with breast cancer (Cohort 2) samples clustering separately from healthy controls (FIG. 1G). Further, when the Z scores of the breast cancer (Cohort 1), lung cancer and melanoma samples were subject to the same clustering analysis, the different cancer types were clearing distinguished from themselves and from healthy controls (FIG. 1F).

These data demonstrate a high degree of representation of tumour gene expression from the bone marrow in the plasma of MDS/AML, breast cancer, lung cancer and melanoma patients. Furthermore, the ability to rank expression enables methods for the determination of gene expression using cfDNA from plasma and distinguish different tumour tissue specific transcriptional profiles.

### Example 2—Gene Expression Determined from cfDNA Shows High Concordance with Tumor Tissue Gene Expression Analysis

To evaluate the performance of the plasma cfDNA method to determine gene expression based on corrected read depth alone (e.g., CPMᵢ), concordance between inferred gene expression profiles using the plasma cfDNA were compared with matched tumour tissue RNA sequencing. In the context of solid malignancies, this comparison is typically challenging due to intra-tumor heterogeneity, such that a single biopsy is unlikely to be representative of all the underlying molecular changes in the cancer, particularly in patients with advanced disease. The scenario is less challenging in the context of haematological malignancies, as the majority of circulating cfDNA in healthy individuals is released from normal hematopoietic cells within the bone marrow compartment. In patients with haematological cancers, such as myelodysplastic syndrome (MDS), ctDNA is released directly into the plasma from the bone marrow compartment. Myeloid neoplasms, such as MDS and acute myeloid leukaemia (AML), show an excellent concordance in mutational profiles between the tumor compartment (bone marrow) and plasma ctDNA, providing an ideal clinical context for the initial evaluation of the plasma cfDNA method.

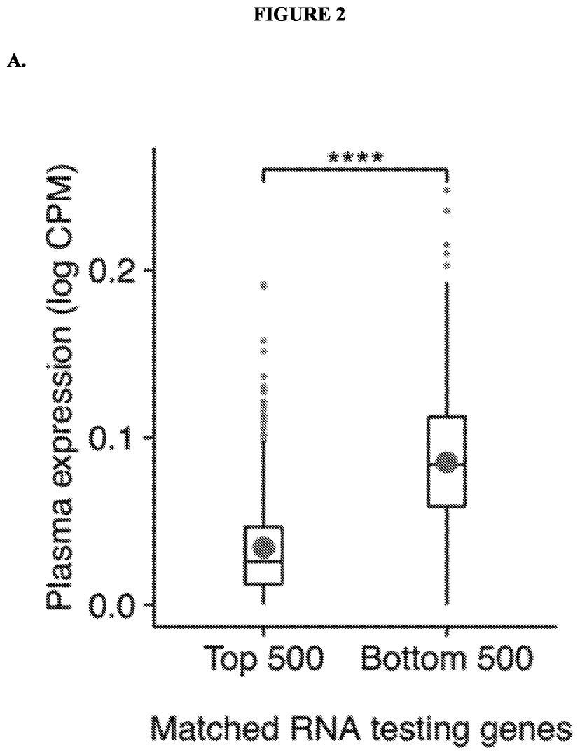

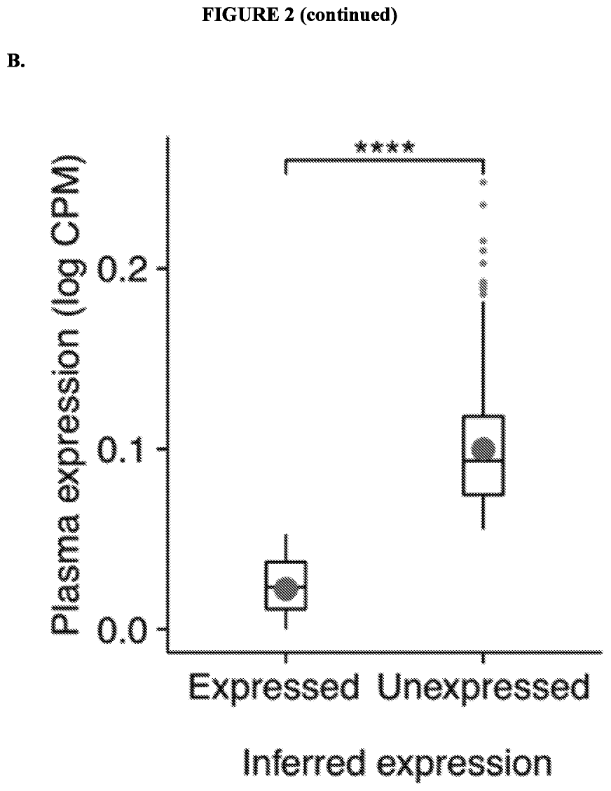

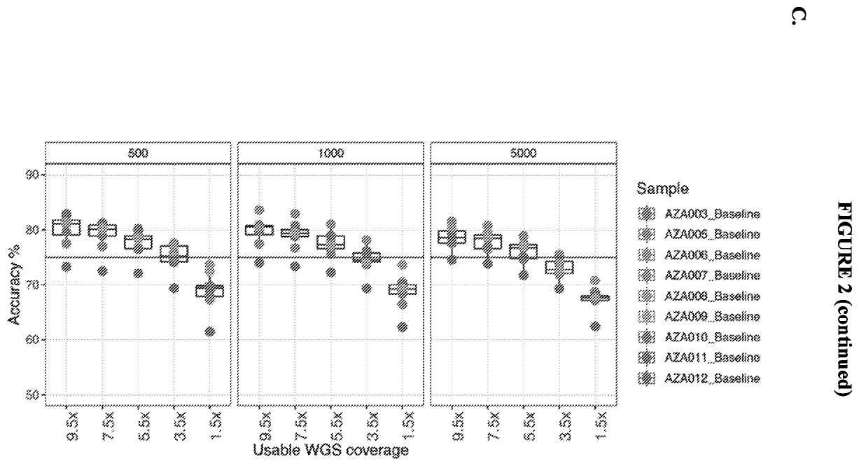

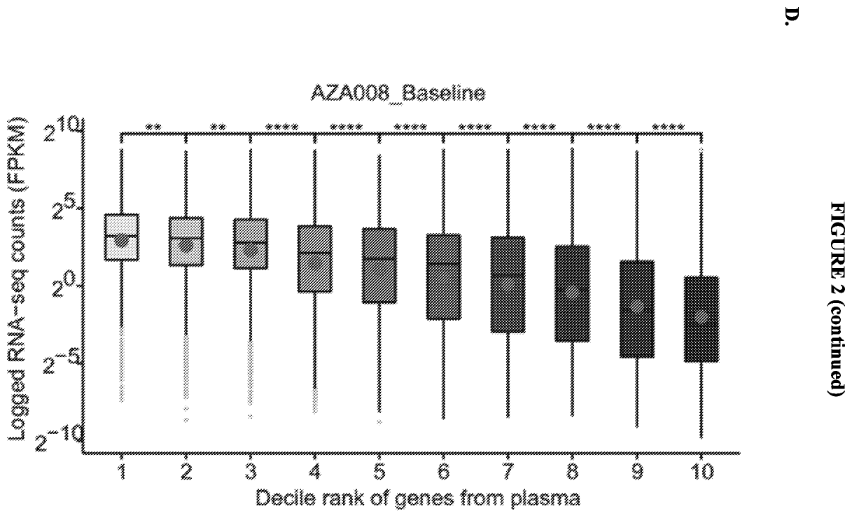

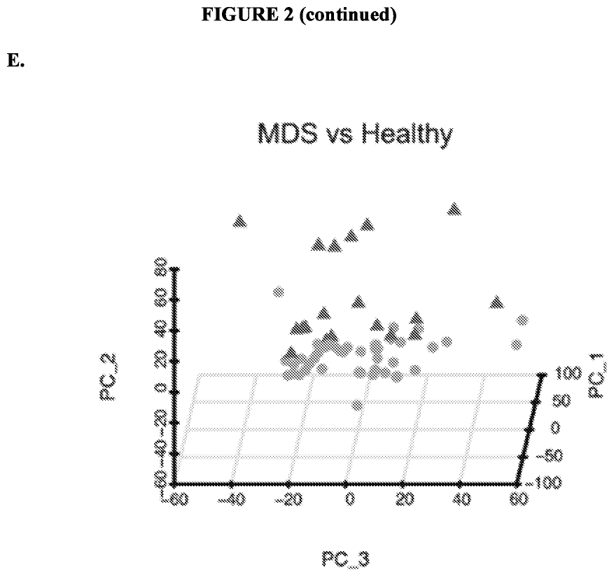

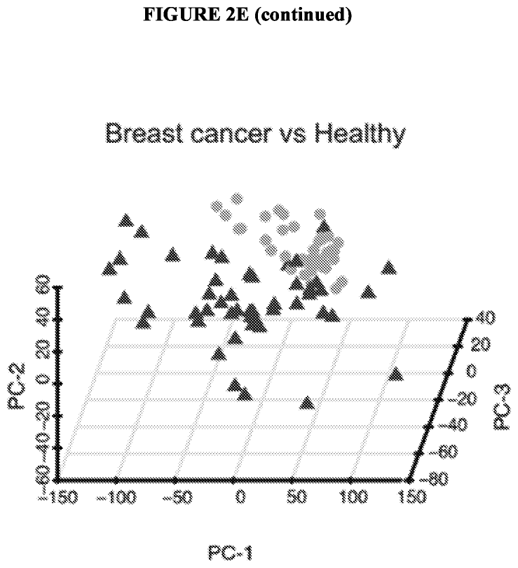

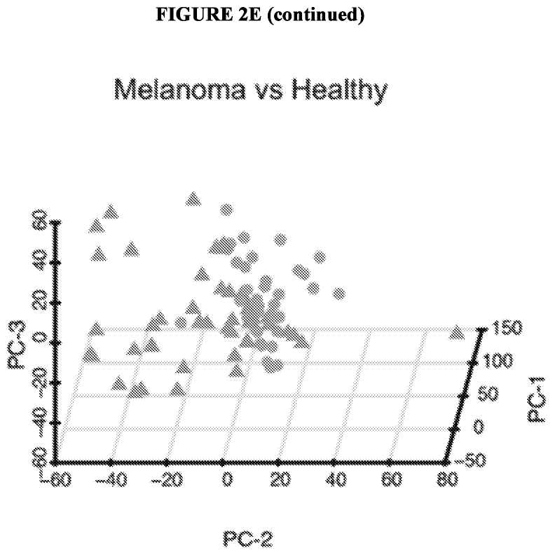

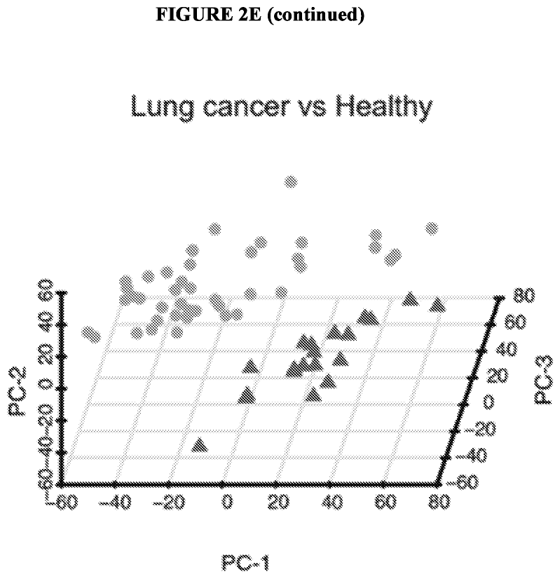

Whole genome sequencing of plasma cfDNA was performed at a median 10× coverage, from patients with MDS prior to treatment. In parallel, full length mRNA-sequencing of matched whole bone marrow aspirate samples was performed as a reference for the tumour gene expression profile. These data showed that plasma cfDNA coverage (i.e., read depth) incrementally decreased over the TSS in an inverse manner, with sequential higher levels of gene expression relevant to such regions determined by RNA-seq from matched bone marrow samples (FIG. 3). Similarly, when the gene expression profile derived from bone marrow RNA-seq was compared to the plasma gene expression profile inferred from the NDR counts generated using the plasma cfDNA method (i.e., corrected read depth), there was a very high degree of concordance of the gene expression profile derived from the RNA-seq data with the inferred gene expression profile generated using the plasma cfDNA method (FIGS. 2A and 2B). Surprisingly, the accuracy of gene expression predictions from plasma analysis was preserved at significantly lower sequencing coverage (3.5×) across multiple patients (FIG. 2C).

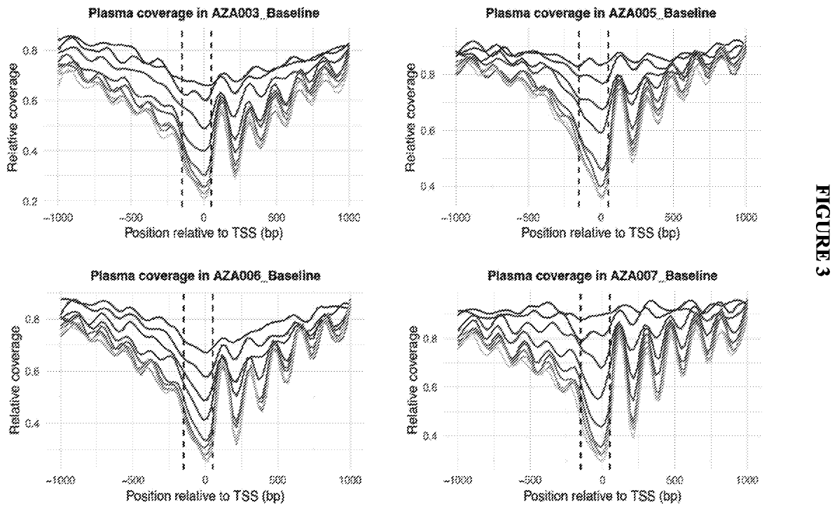

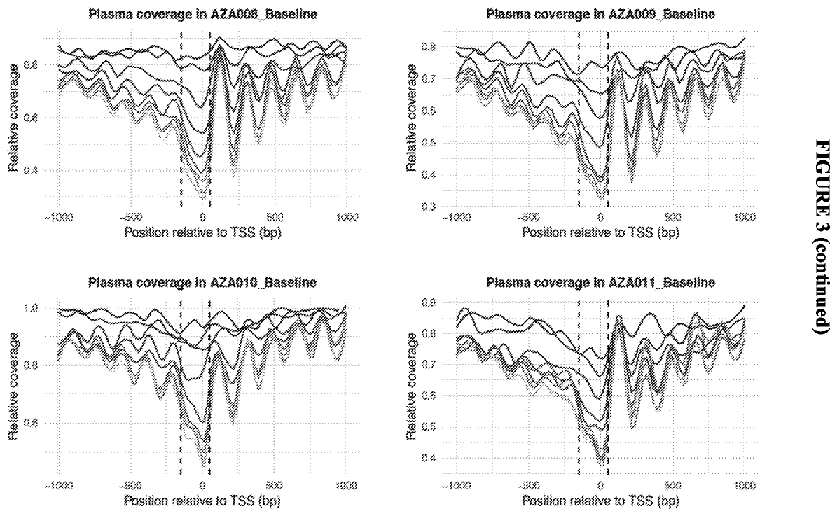

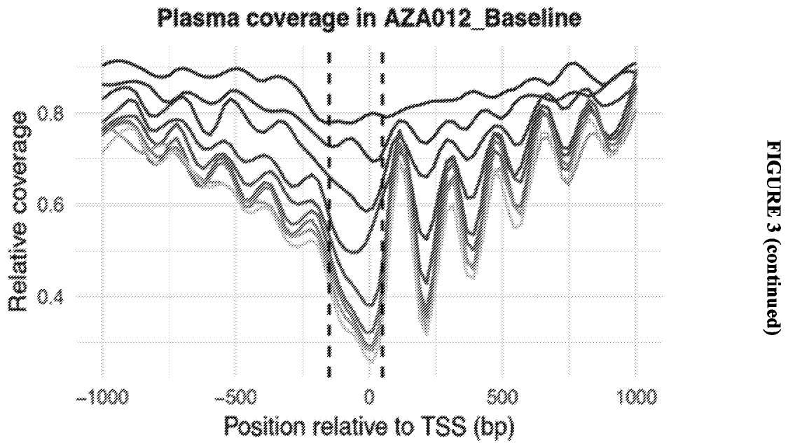

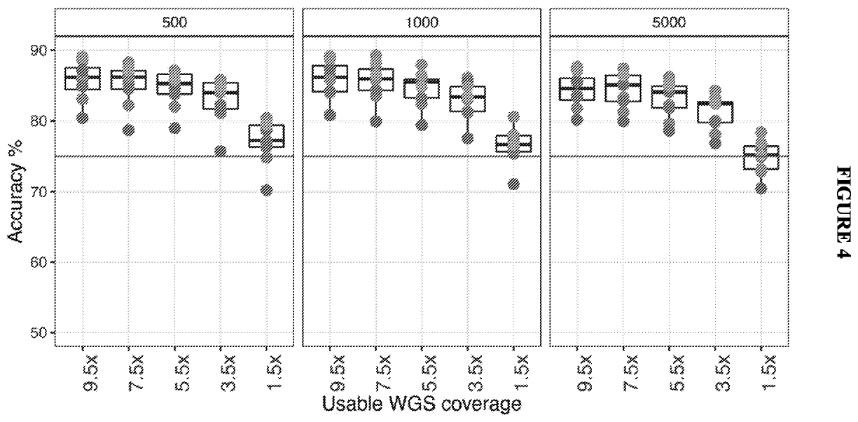

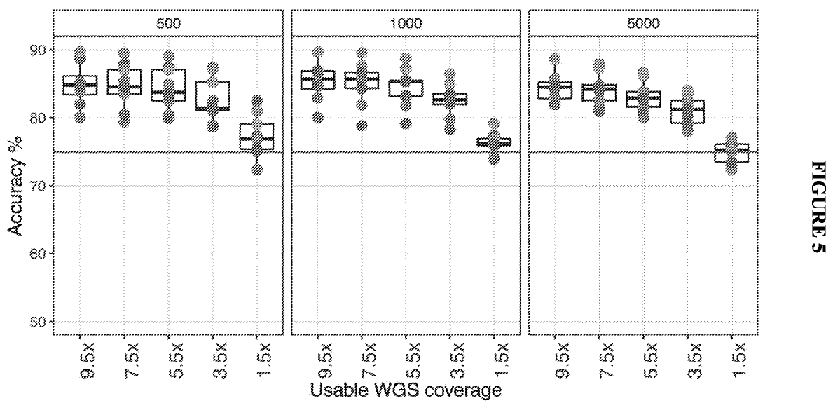

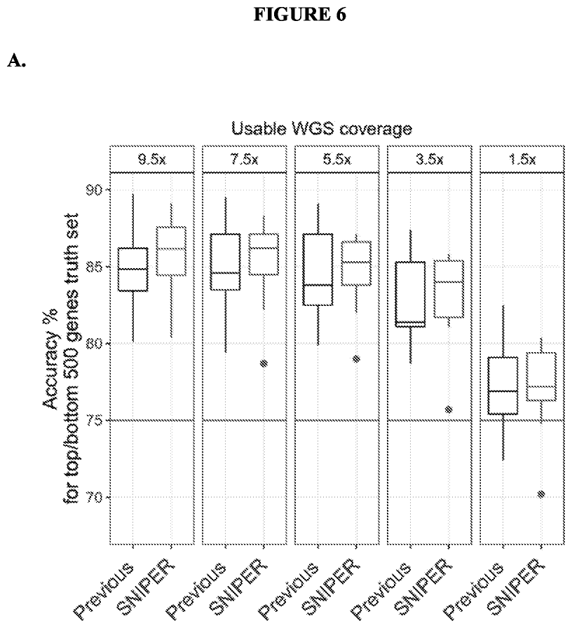

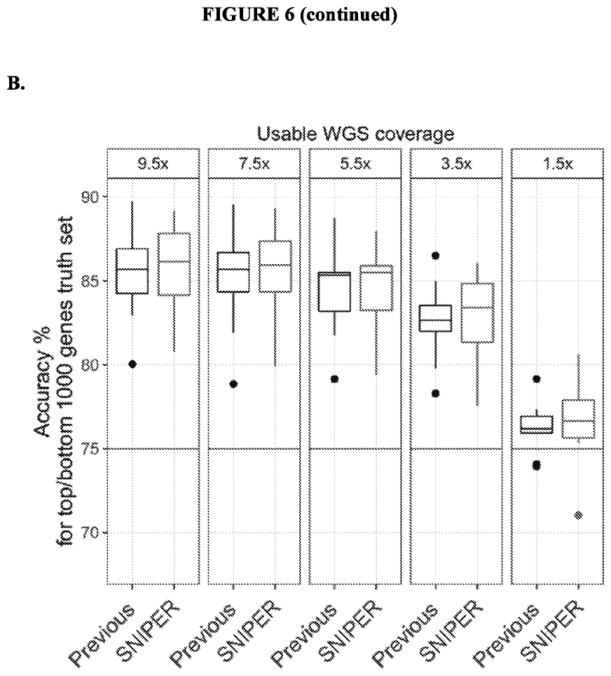

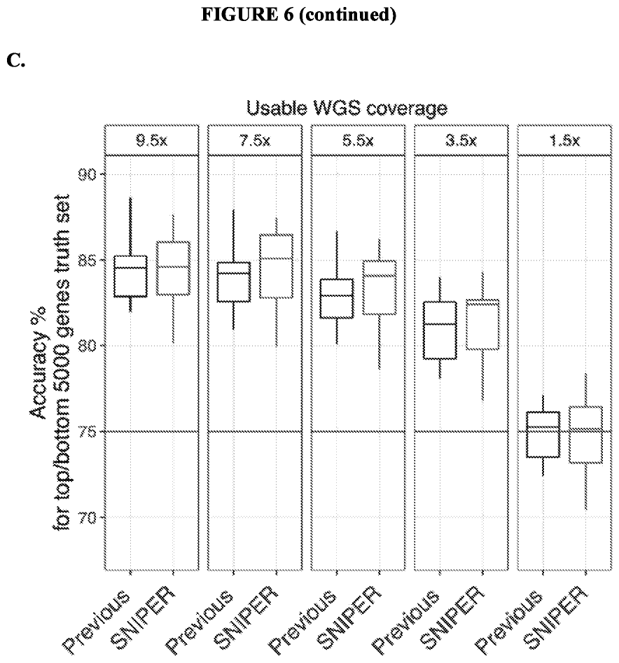

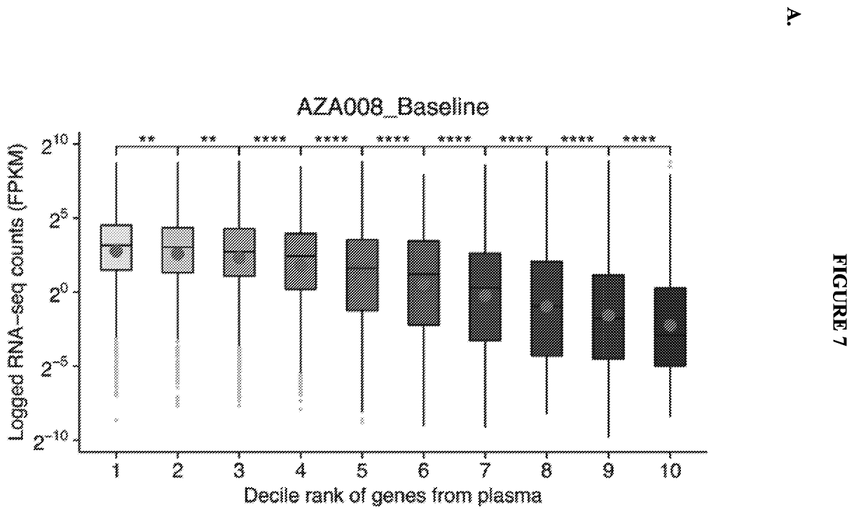

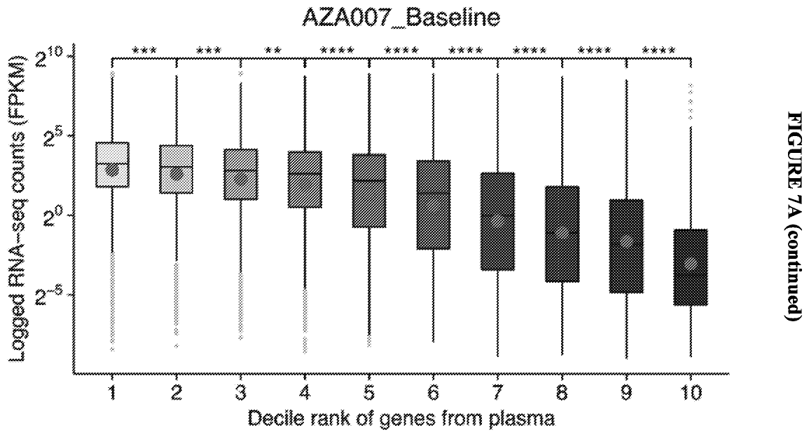

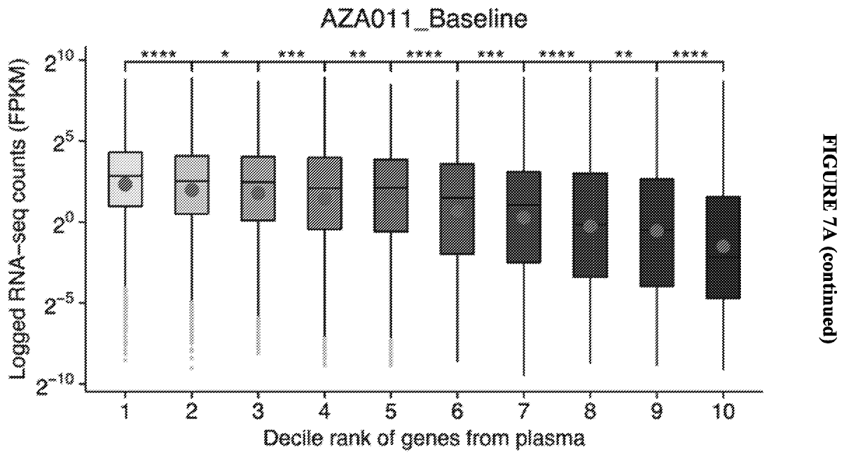

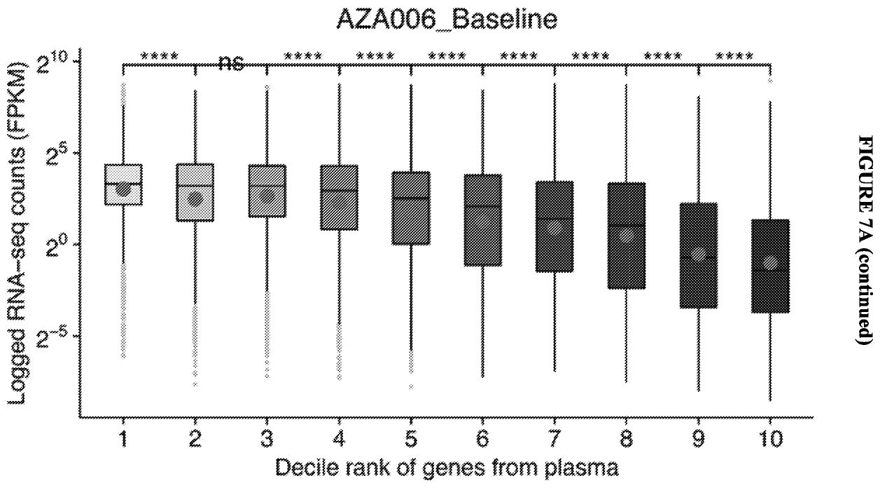

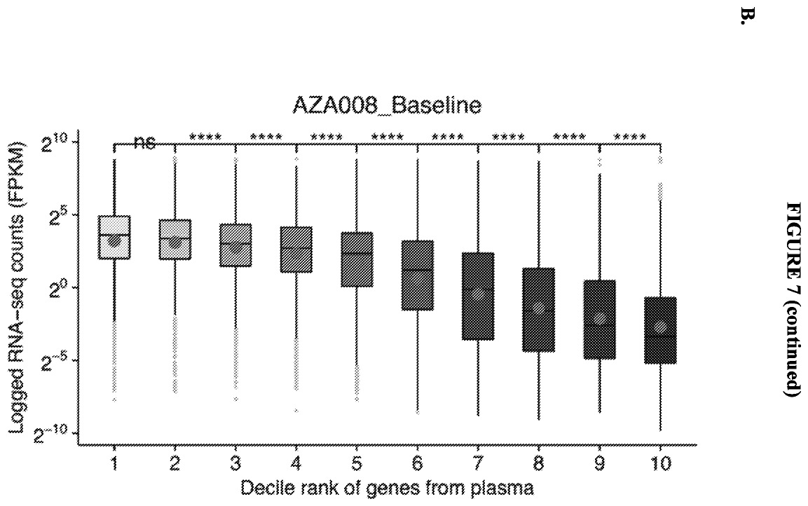

Other methods that have sought to utilise ctDNA for determining gene expression have been limited to a binary assessment of genes as being expressed or non-expressed. For example, the method of Ulz et al., (2016, supra) is two feature classifier that is effective for the binary assessment of gene expression. A head-to-head analysis of the plasma cfDNA method described herein against the method of Ulz et al. on the above dataset demonstrated that the plasma cfDNA method displayed either the same or improved median accuracy across multiple patients at the different depths of coverage tested (FIGS. 4-6). These data show that the plasma cfDNA method described herein is highly accurate in binary gene expression predictions from plasma.

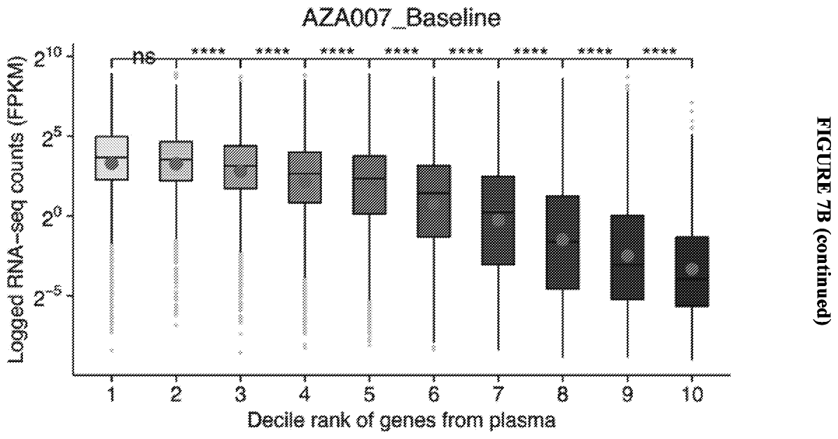

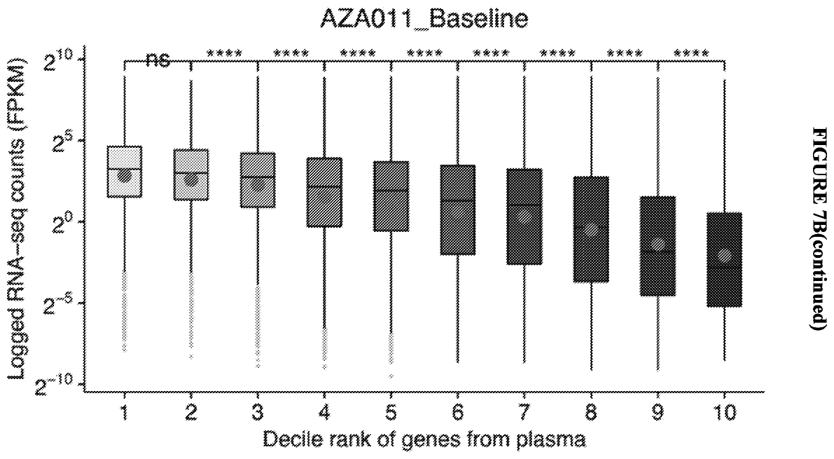

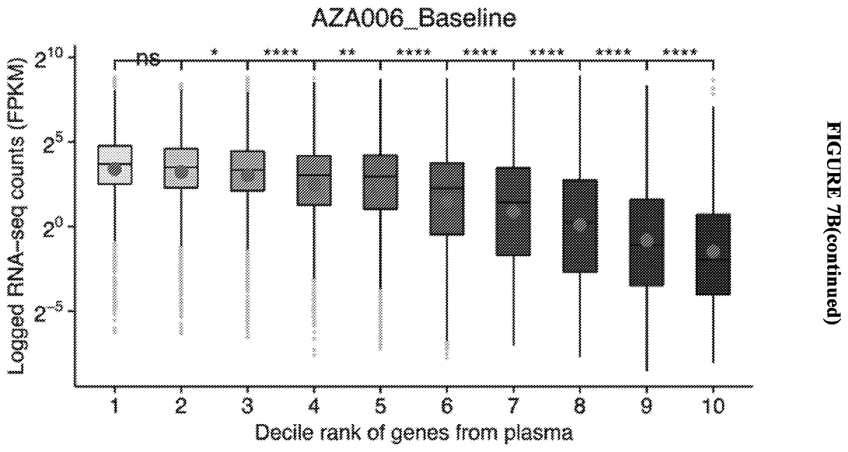

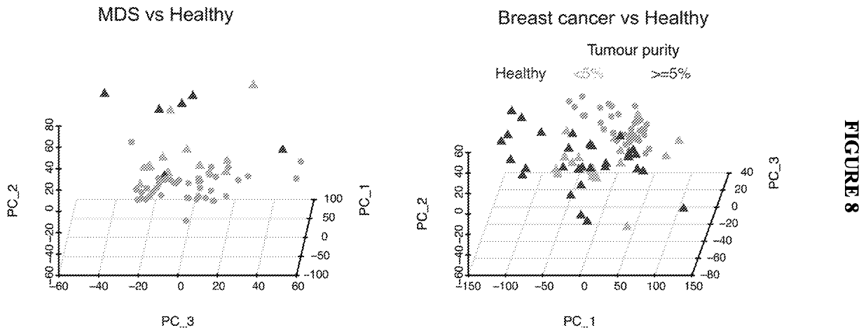

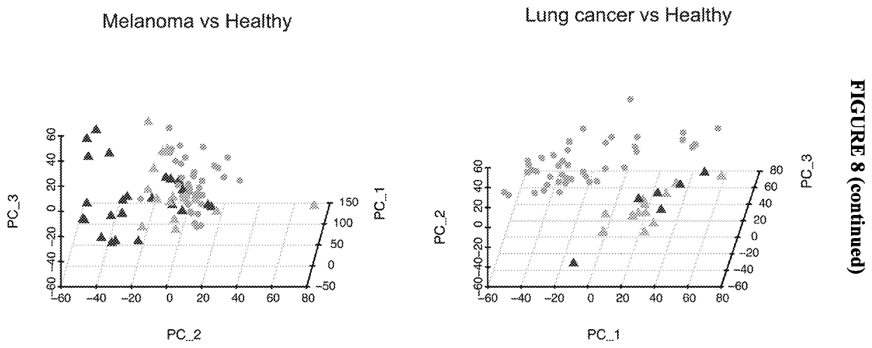

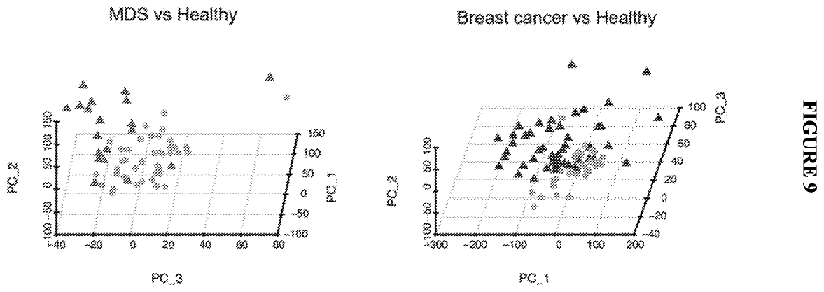

However, quantitative assessment of gene expression is required for serial transcriptional monitoring and analysis of enriched biological pathways. Therefore, we investigated the use of plasma cfDNA method to provide information on the level of gene expression rather than simply categorising a gene as either expressed or unexpressed. Here, plasma cfDNA method clearly differs from previous methods, e.g., Ulz et al., 2016, supra, by capturing the dynamic range of gene expression, which recapitulated gene expression levels from matched tumor tissue RNA-seq (FIG. 2D). The coverage signal (i.e., read depth) generated for the flanking region comprising the NDR (i.e., −150 bp to +50 bp with respect to the TSS) gave more power in discriminating the most highly expressed genes, when compared to the counts from the wider region +/−1000 bp with respect to the TSS (i.e., 2K_TSS). While the dynamic quantitation of gene expression could be determined using either the flanking regions comprising the NDR or the 2K_TSS region, the use of both coverage signals was more effective to categorise gene expression levels (FIG. 7).

Taken together, these data enable the use of the plasma cfDNA method to infer gene expression and the dynamic quantitation of gene expression levels from cfDNA. Further analysis of the plasma cfDNA method based on corrected read depth (e.g., CPMᵢ) is described in Examples 3 to 6.

### Example 3—Gene Expression Determined from Plasma cfDNA Reliably Detects Tumor Specific Gene Expression Profiles

Given that the majority of cfDNA is ordinarily derived from haematopoietic cells, we evaluated if the plasma cfDNA method could distinguish malignant haematopoietic gene expression from normal haematopoietic gene expression. Through unsupervised clustering of corrected read depth (e.g., CPMᵢ), the plasma cfDNA method identified specific malignant haematopoietic transcriptional changes in patients with MDS compared to healthy individuals (FIG. 2E).

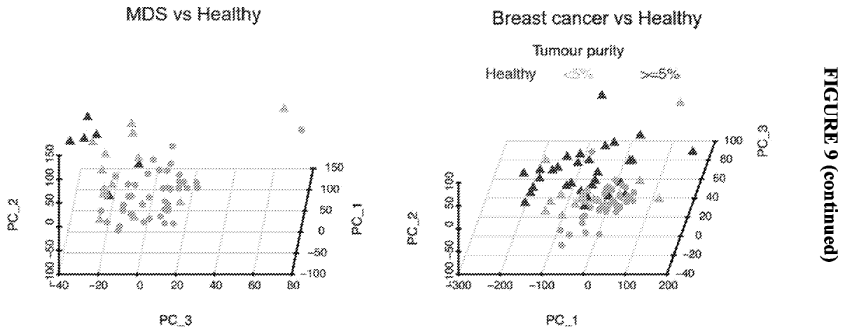

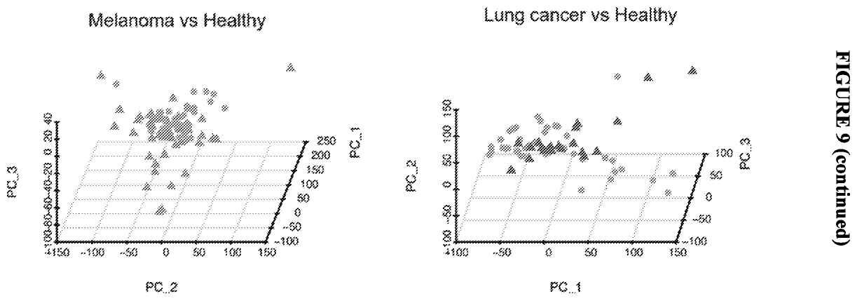

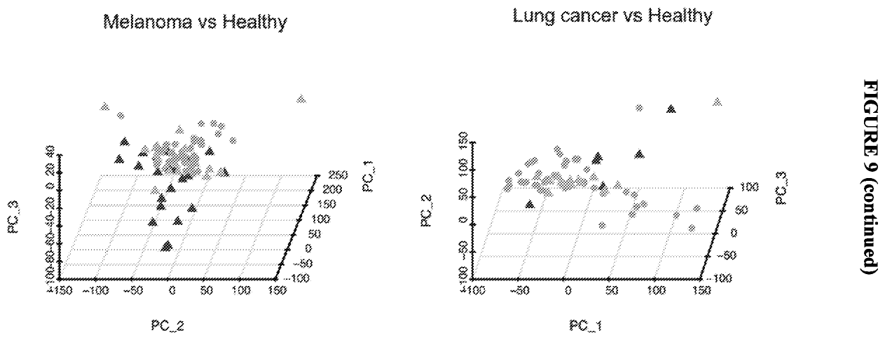

We also assessed whether the plasma cfDNA method could infer tissue-specific transcriptional profiles across a range of common solid cancers. When the plasma cfDNA method was applied to whole genome sequencing of plasma samples from individuals with advanced estrogen receptor (ER) positive breast cancer, EGFR mutant non-small cell lung cancer (NSCLC) and BRAF mutant melanoma, unsupervised clustering showed clear separation between healthy and cancer samples across the different cohorts according to the inferred gene expression profiles. The plasma cfDNA method distinguished cancer versus healthy samples, even in cases with low ctDNA fractions (FIG. 2E). The corrected read depth derived from genomic regions comprising the NDR provided the optimal region for the plasma cfDNA method (FIG. 8) as it provided greater separation of cancer versus healthy samples compared to using the wider 2K_TSS region (FIG. 9).

These data enable the use of corrected read depth (e.g., CPMᵢ) to infer tumour-specific gene expression profiles across both solid and haematological malignancies in different plasma-derived ctDNA fractions.

### Example 4—Gene Expression Determined from cfDNA can be Used to Characterise Key Upregulated Cancer Pathways

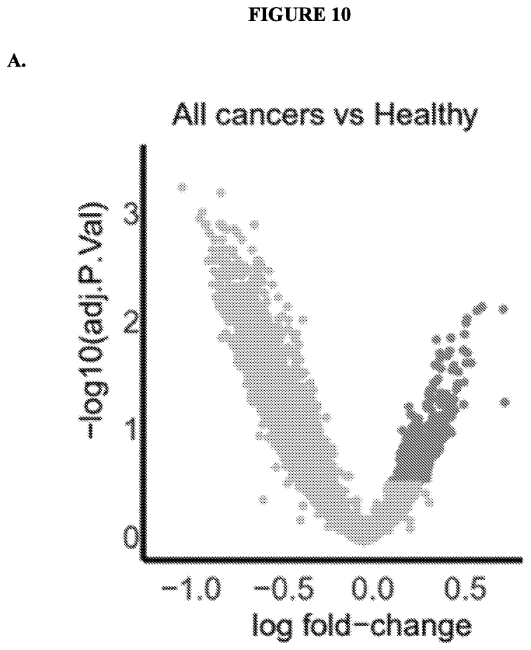

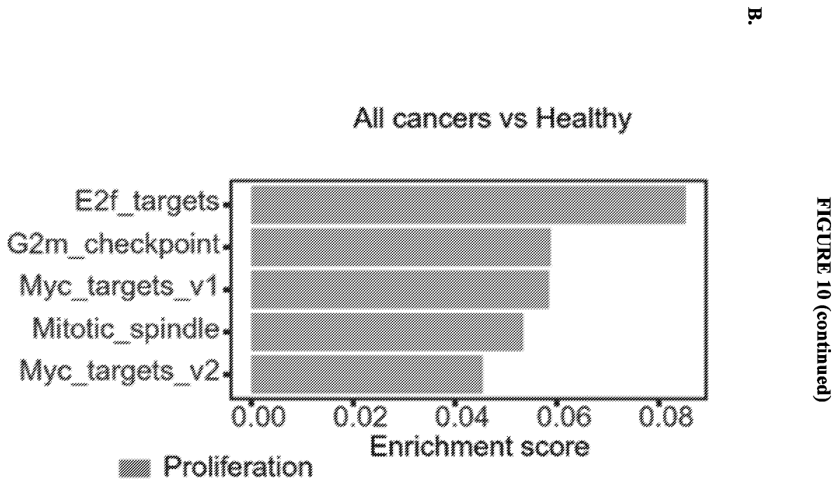

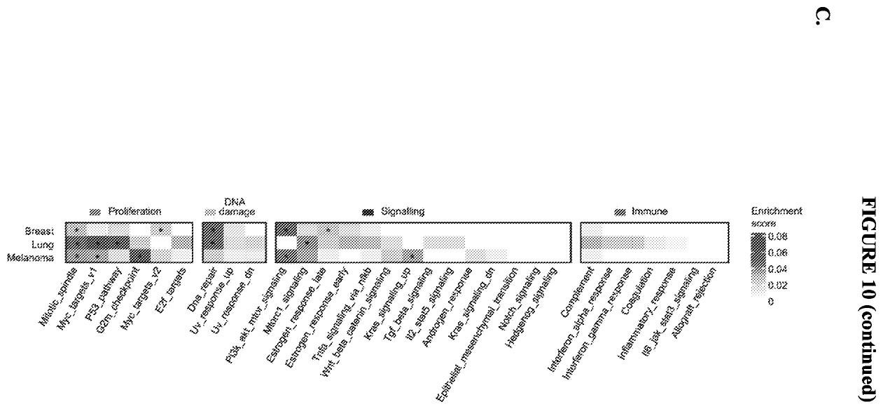

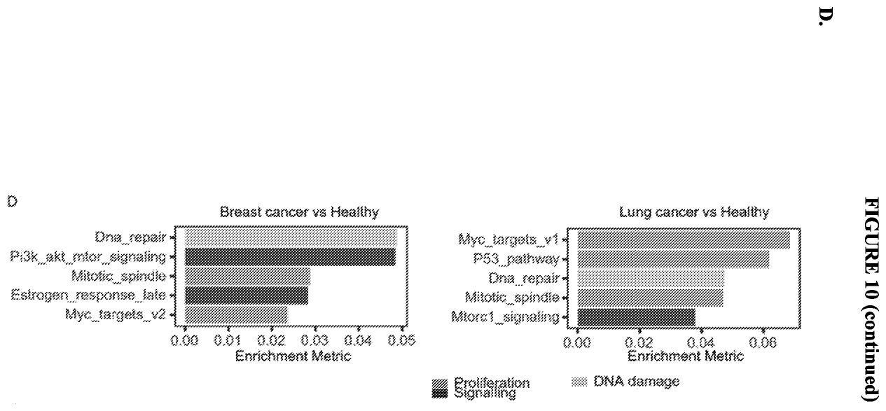

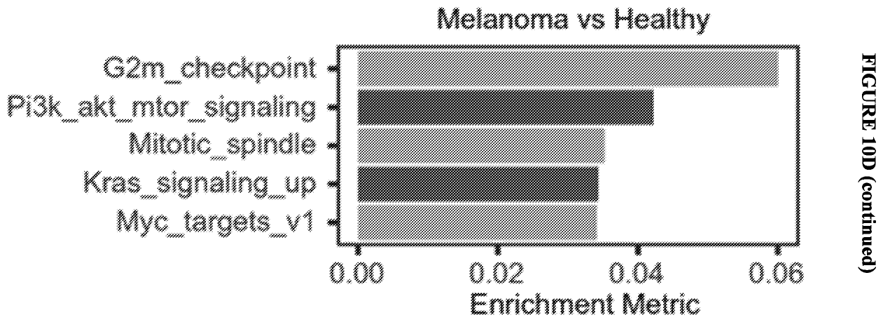

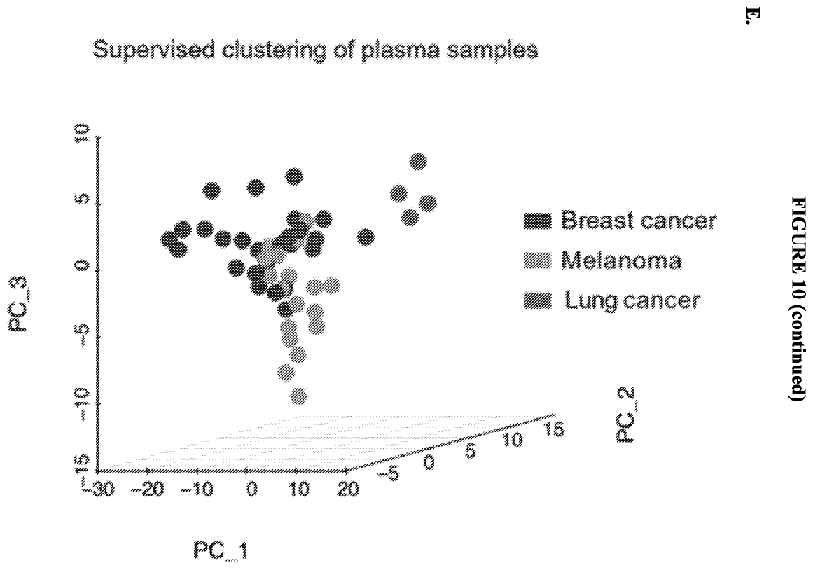

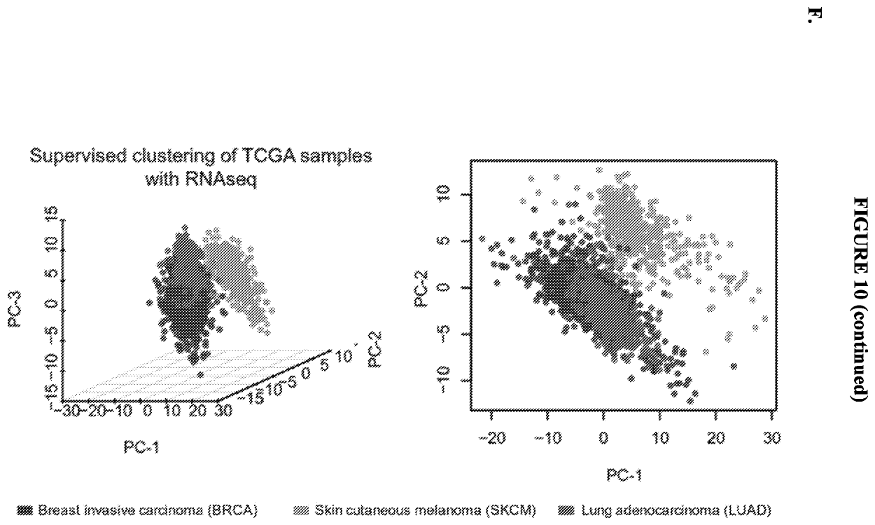

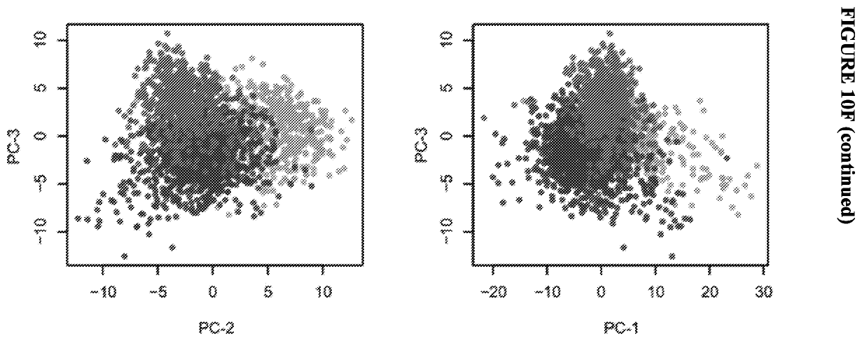

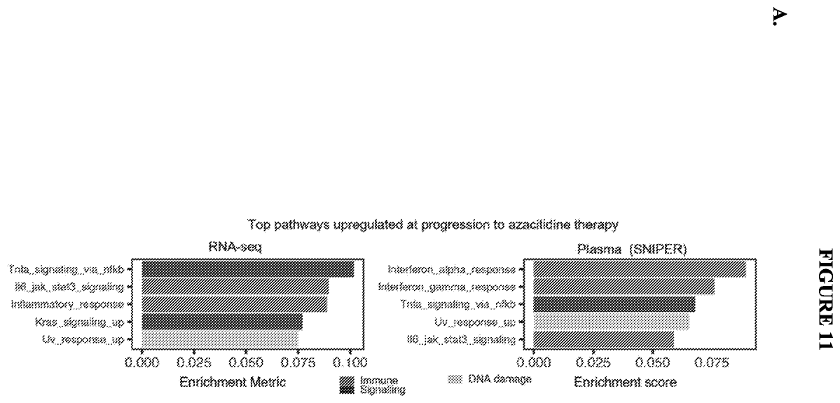

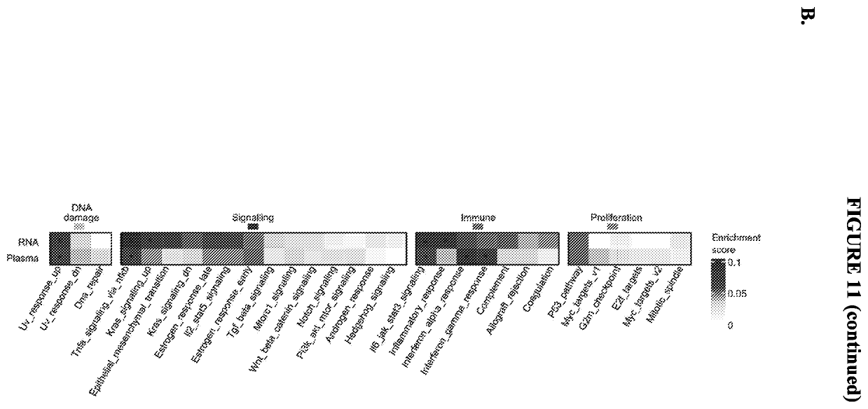

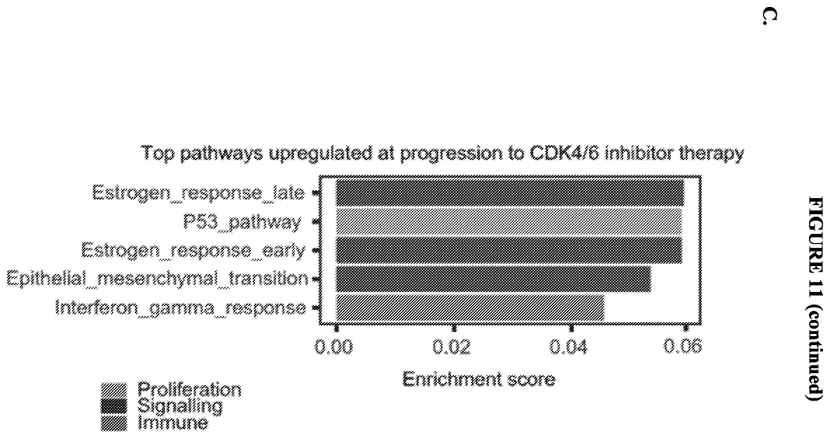

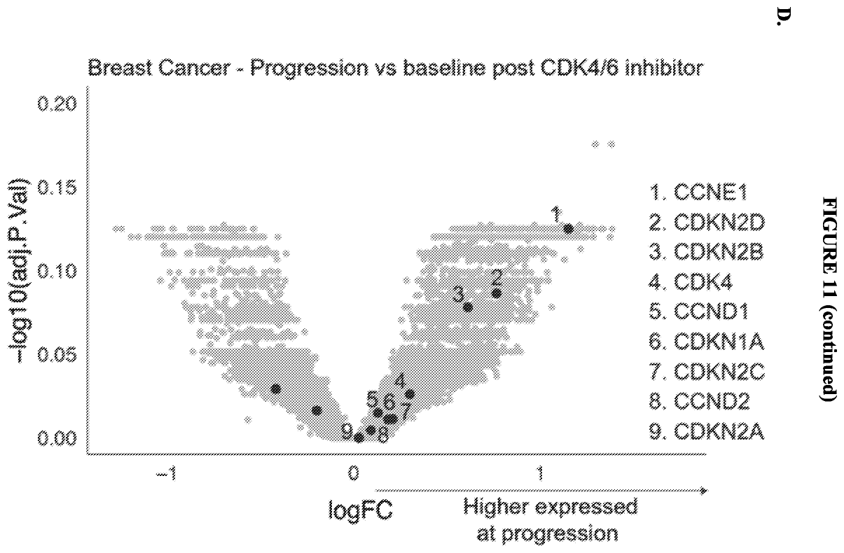

The most highly expressed genes across our cancer and healthy plasma samples were examined to identify upregulation of key oncogenic pathways using the plasma cfDNA method. Following this analysis, the biological pathways most enriched were those associated with cellular proliferation, which was a consistent pattern observed across all samples (FIG. 10A).

To enhance the tumour-type specific gene expression signal from ctDNA compared to the background common gene expression signature, genes that were highly expressed across all healthy controls and cancer samples were filtered, regardless of tumour type. Following filtering of this background gene signature, the top expressed genes within each cancer type were defined and analysed for enrichment of HALLMARK pathway-specific genes. This analysis identified key oncogenic signalling pathways that were upregulated and distinct in each cancer type, including enrichment of PI3K/AKT/mTOR signalling and estrogen response genes in ER positive breast cancer, upregulation of KRAS signalling in BRAF mutant melanoma, and upregulation of p53 signalling in the EGFR mutant NSCLC cohort (FIGS. 10B and 10C), confirming the ability of the plasma cfDNA method to characterise distinct oncogenic signalling pathways that are upregulated in different tumour types.

To further validate the tumour specificity of the plasma cfDNA method, the differences between the tumour type-specific transcriptional profiles were evaluated using supervised clustering. The plasma cfDNA method generated tumour-specific gene sets distinguished the solid malignancies based on tumour type (FIG. 10D). Furthermore, the tumour-specific gene sets were applied to the TCGA Tumour RNA-seq dataset, which demonstrated consistent upregulation of the tumour type-specific transcriptional profiles across each of the solid malignancies (FIG. 10H).

Together, these data enable the use of the plasma cfDNA method described herein for evaluating tumour type-specific transcriptional profiles from plasma-derived ctDNA.

### Example 5—Gene Expression Determined Using cfDNA Facilitates Non-Invasive, Serial Monitoring of Transcriptional Evolution Following Therapy

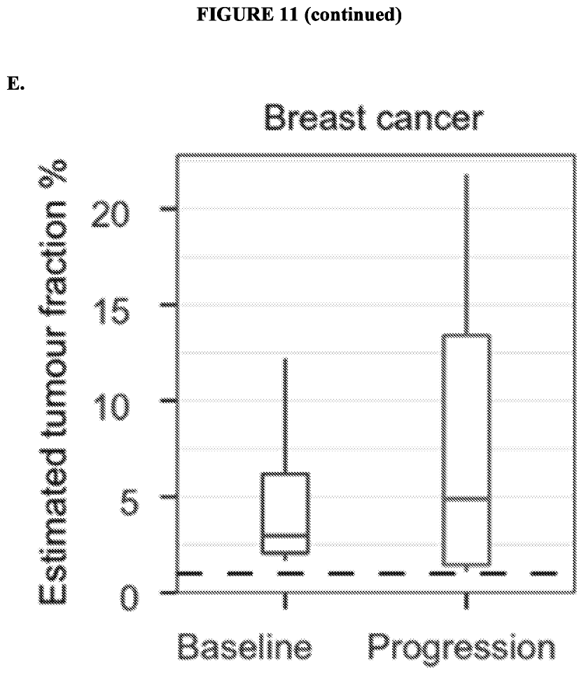

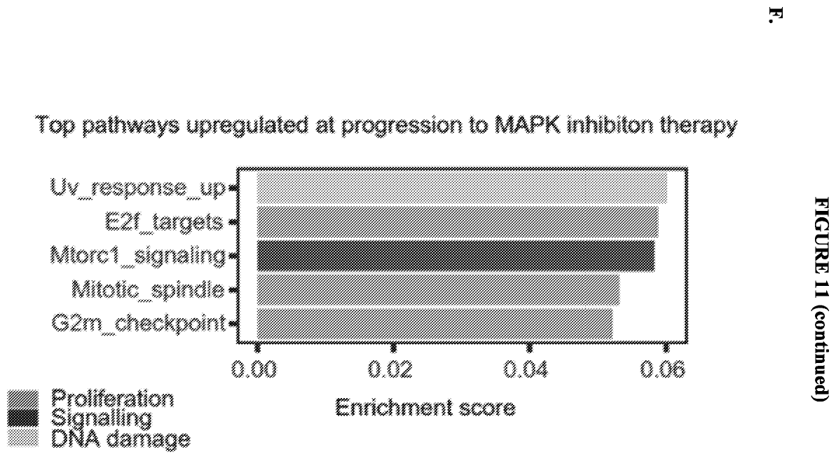

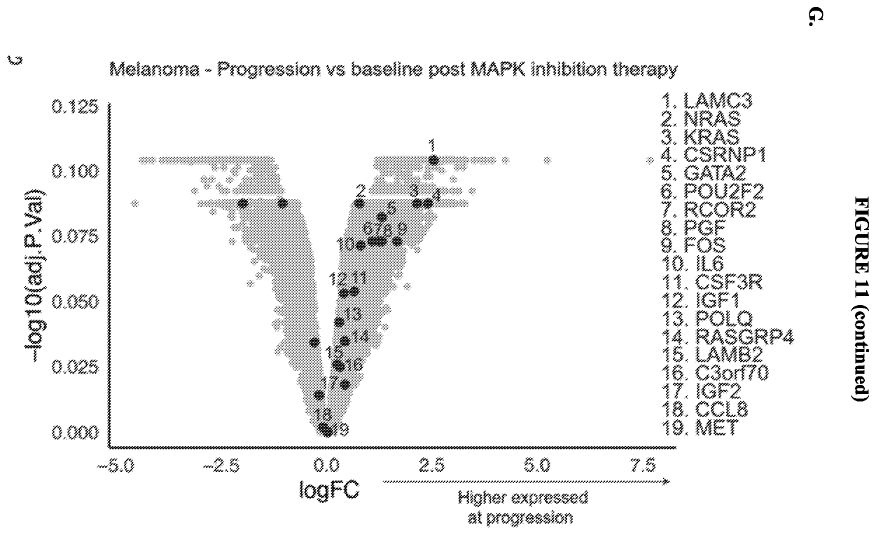

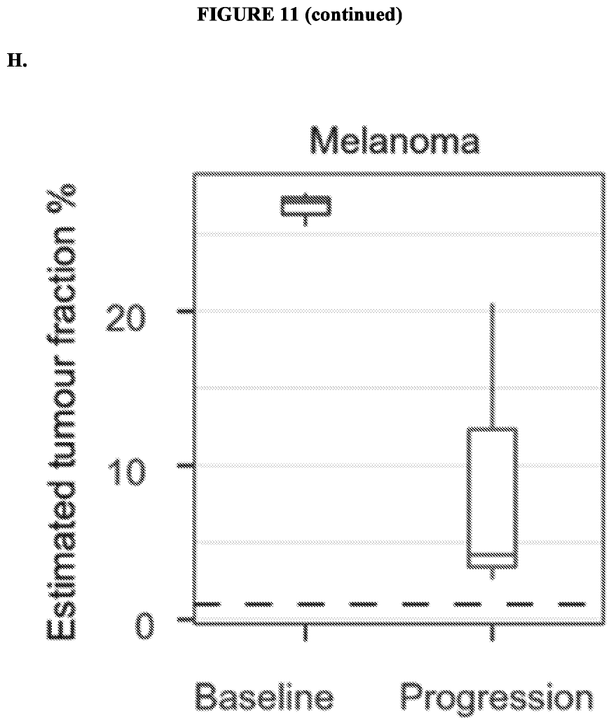

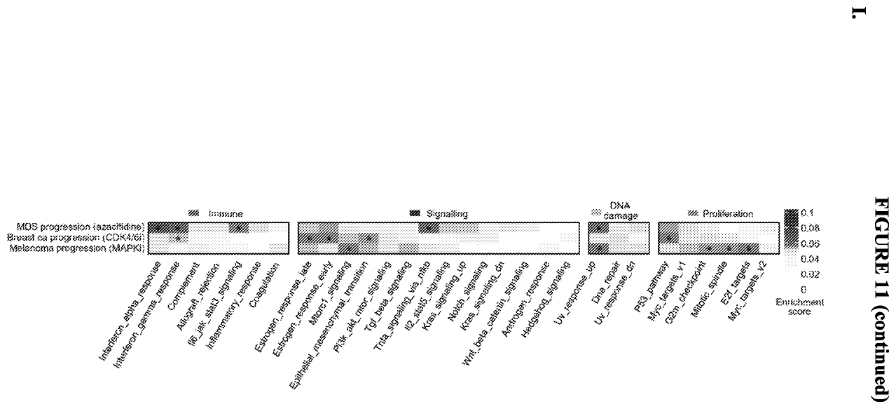

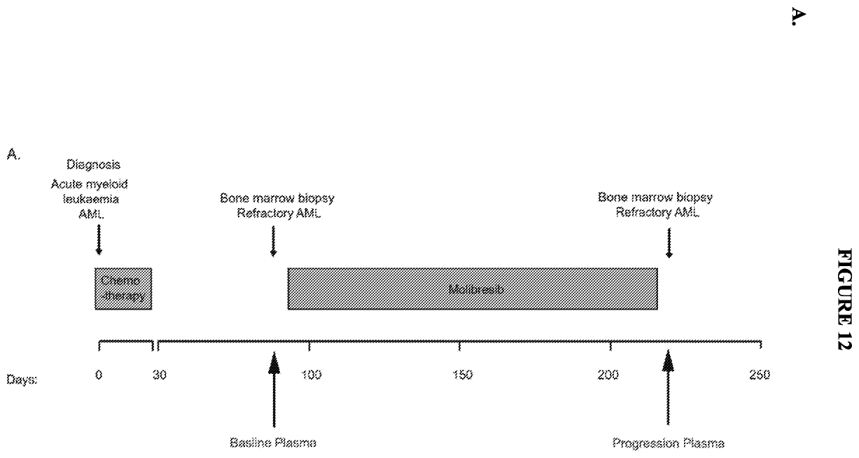

To investigate the ability for gene expression determined using cfDNA to serially monitor transcriptional evolution during cancer therapy, concordance between tumour tissue profiling and the plasma cfDNA method was assessed in the MDS patient cohort treated with the DNA hypomethylating agent, azacitidine. Differential gene expression analysis was performed between tumour tissue samples analysed by RNA-seq at the time of disease progression, compared to baseline (i.e., prior to azacitidine therapy) and pathway enrichment analysis was performed on a subset of overexpressed genes at progression. The same analysis was performed on temporally matched plasma samples collected at the time of progression, compared to baseline, after adjusting for the changes in ctDNA fraction of each patient across the two time points. Surprisingly, there was high concordance between the analysis performed using the standard tumour tissue analysis method and the plasma cfDNA method at the pathway level, including enrichment of key immune and inflammatory signalling pathways (FIGS. 11A and 11B). These data are consistent with previous studies, such as Unnikrishnan et al. (2017, Cell Reports, 20:572-585 and Roulois et al. (2015, Cell, 162:961-973), which have shown increased inflammatory response and interferon signalling in response to DNA methyltransferase (DNMT) inhibition by reducing methylation of endogenous retroviral genes, thereby triggering a viral mimicry response to activate interferon signalling genes.

The plasma cfDNA method was then assessed for serial transcriptional profiling in the context of advanced ER positive breast cancer patients who received uniform therapy with the CDK4/6 inhibitor, palbociclib, and an aromatase inhibitor, letrozole. The plasma cfDNA method distinguished evolving gene expression signatures between baseline and progression on palbociclib and letrozole therapy. An enrichment of several important pathways, including persistent upregulation of estrogen response genes, p53 and interferon-Y signalling was also observed following treatment (FIG. 11C). Importantly, CDK4/6 inhibitors have been shown to enhance interferon driven gene expression programs to promote anti-tumour immunity. Due to limitations in access to fresh breast cancer tissue (e.g., by serial biopsy), it is difficult to characterise patterns of transcriptional adaptation to CDK4/6 inhibitor therapy in breast cancer using clinical samples. The NeoPalAna clinical trial investigated the role of neoadjuvant palbociclib and anastrozole in ER positive early-stage breast cancer. Tumour tissue biopsies were performed at baseline and following neoadjuvant therapy and gene expression profiling was performed using microarray analysis. Importantly, this clinical study showed an association between resistance and persistent on-treatment expression of E2F targets, including CCNE1 and CDKN2D. The genes found to be upregulated in NeoPalAna, closely overlapped with upregulated genes determined using the SNIPER method, providing orthogonal validation for these data (FIG. 11D). Finally, high CCNE1 expression has recently been identified as a predictive biomarker of relative resistance to palbociclib in the large phase III Paloma-3 trial in which metastatic breast cancer patients received treatment with palbociclib and fulvestrant or palbociclib alone. Clinical studies to date have revealed very few genetic changes that have been associated with resistance to CDK4/6 inhibitors, other than the rare event of RBI loss. The plasma cfDNA method was able to detect such changes in plasma samples with a tumour fraction of ≤3% (FIG. 11E).

Serial samples of patients with advanced BRAF mutant melanoma undergoing treatment with combination BRAF (dabrafenib) and MEK inhibitor (trametinib) therapies blocking the MAPK pathway were also analysed using the plasma cfDNA method. Consistent with prior tumour tissue studies exploring non-genomic evolution of melanomas acquiring MAPK inhibitor resistance, the plasma cfDNA method demonstrated upregulation of key proliferation and signalling pathways, including mTOR, which is downstream of PI3K/AKT and is a recognised mechanism of MAPK inhibitor drug resistance (FIG. 11F). These data also overlapped with key pathways identified in a previously published dataset of genes overexpressed in melanoma tumours with resistance to MAPK inhibitor therapy (Hugo et al., 2015, Cell, 162:1271-1285). This included upregulation of NRAS, KRAS and FOS, a tumour cell intrinsic transcriptional factor activated downstream of MAPK, which has previously been implicated in MAPK inhibitor resistance (FIG. 11G). Although the average estimated ctDNA fraction was higher at baseline compared to the breast cancer cohort, the ctDNA fraction in the progression samples was significantly lower (FIG. 11H). Despite this, the plasma cfDNA method was able to detect the highly specific upregulation of NRAS and KRAS in progression plasma samples within the melanoma cohort.

Together, these data enable the use of the plasma cfDNA method described herein to capture serial transcriptional changes following treatment across different malignancies and to monitor non-genomic evolution in the context of acquired resistance to cancer therapy.

### Example 6—Gene Expression Determined from cfDNA can Uncover Transcriptional Adaptation Resulting in Treatment Resistance

In the setting of haematological malignancies, MDS frequently progresses to AML. This evolution is non-linear, due to a large reservoir of genetically and transcriptionally diverse malignant stem cells. As bulk genomic and transcriptomic data does not have sufficient resolution to identify the molecular mechanisms of progression, single cell technologies have been used to gain greater insights into the molecular pathogenesis of AML. However, these approaches can be both technically challenging and resource intensive.

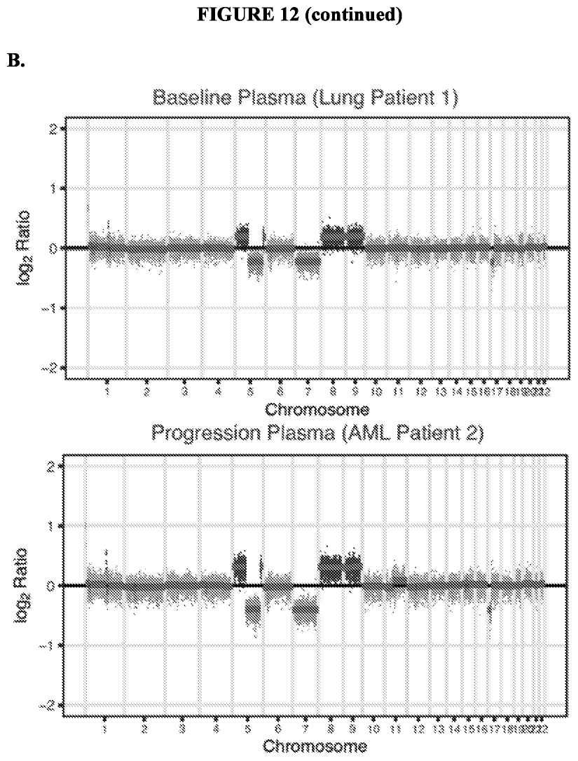

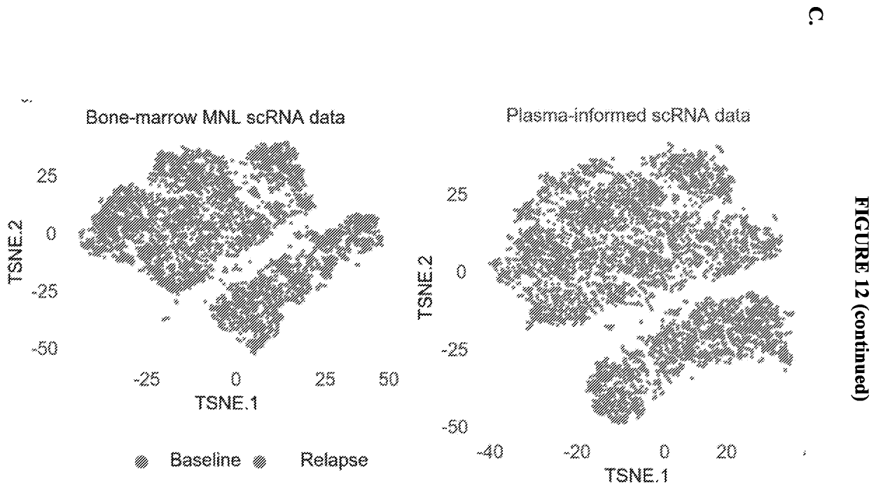

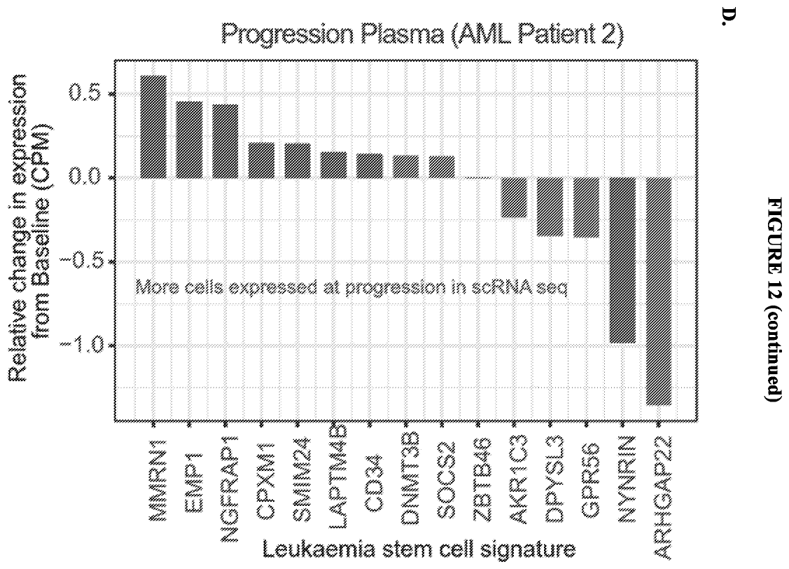

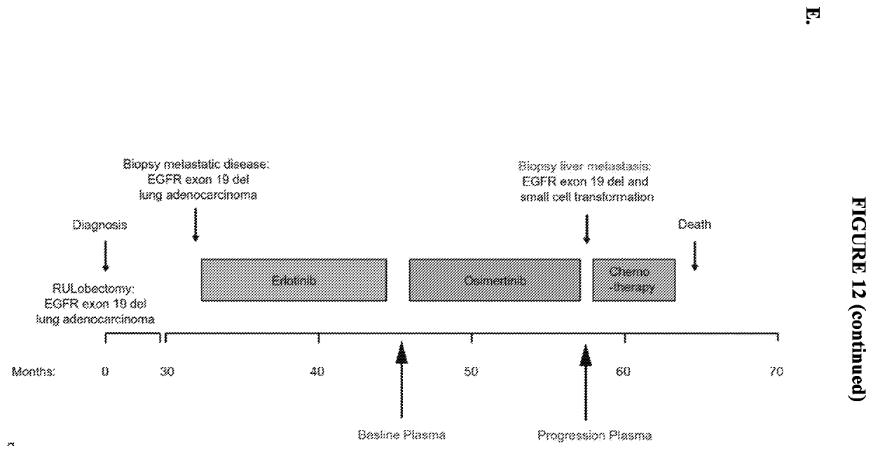

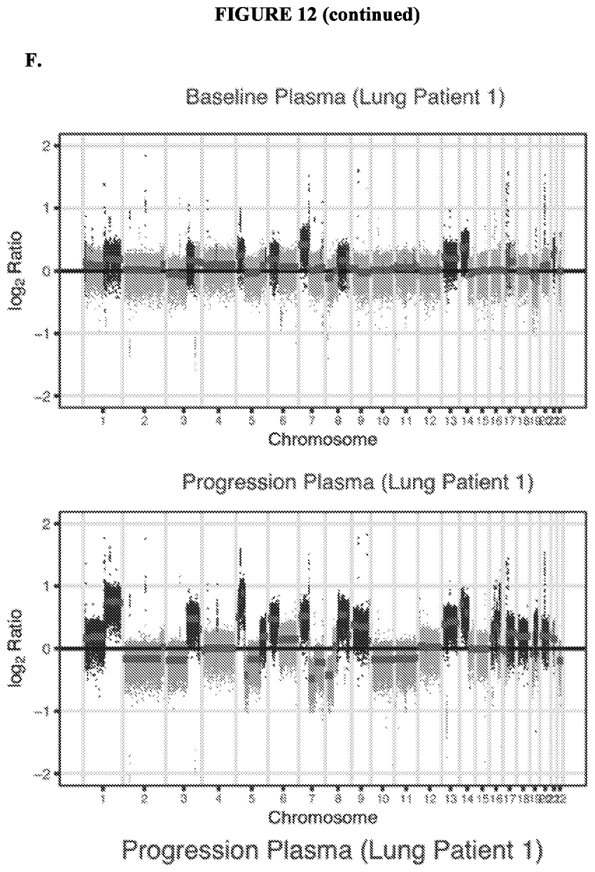

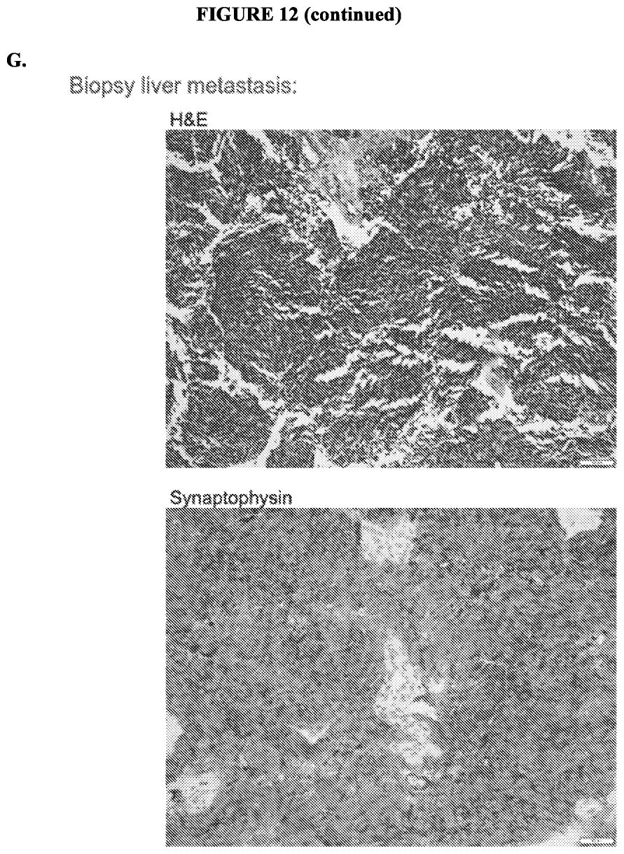

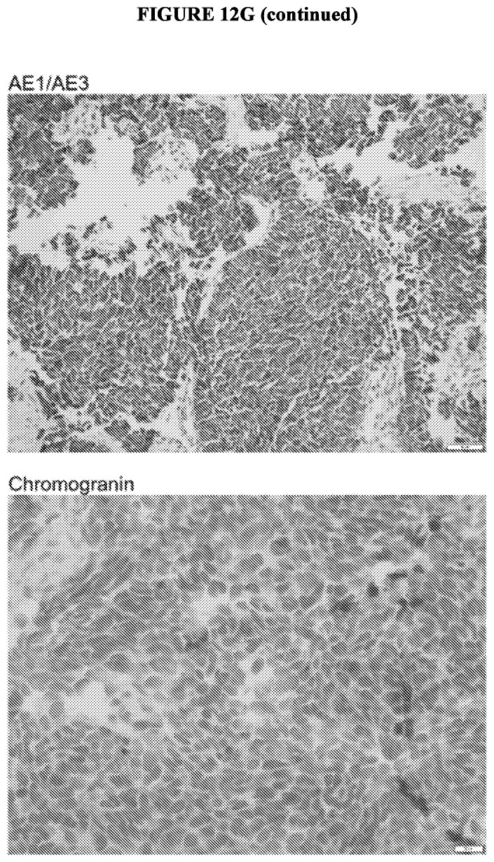

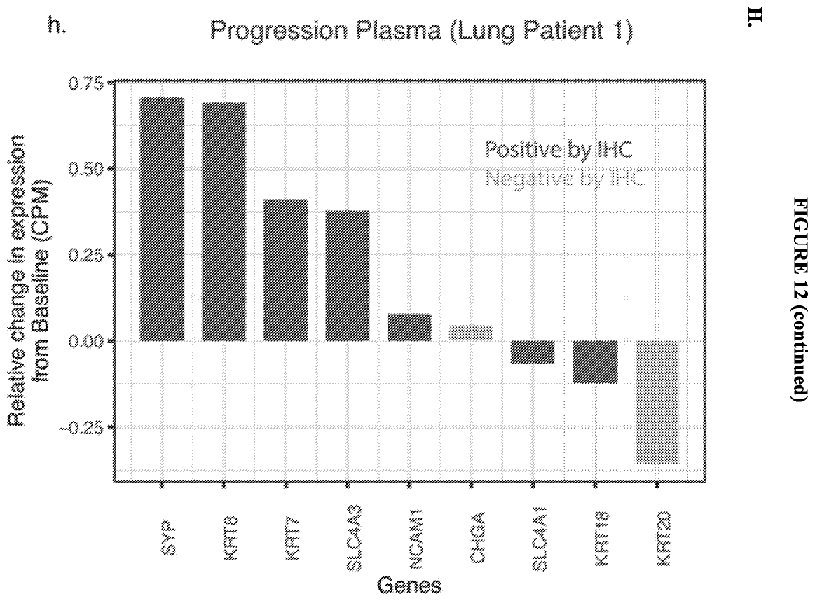

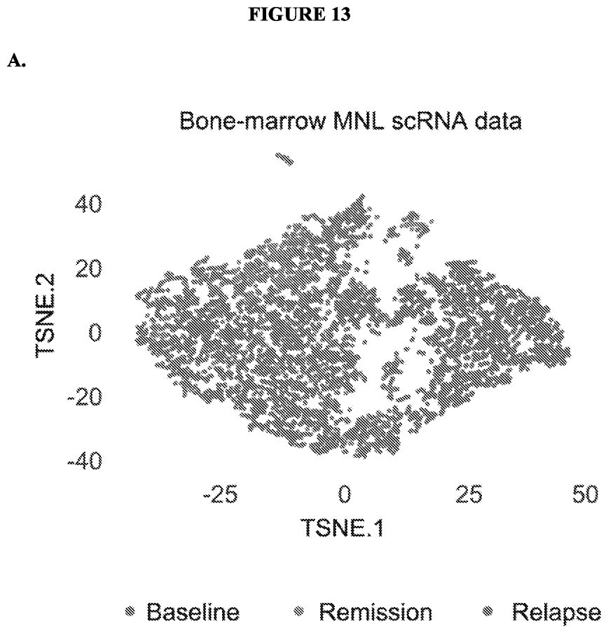

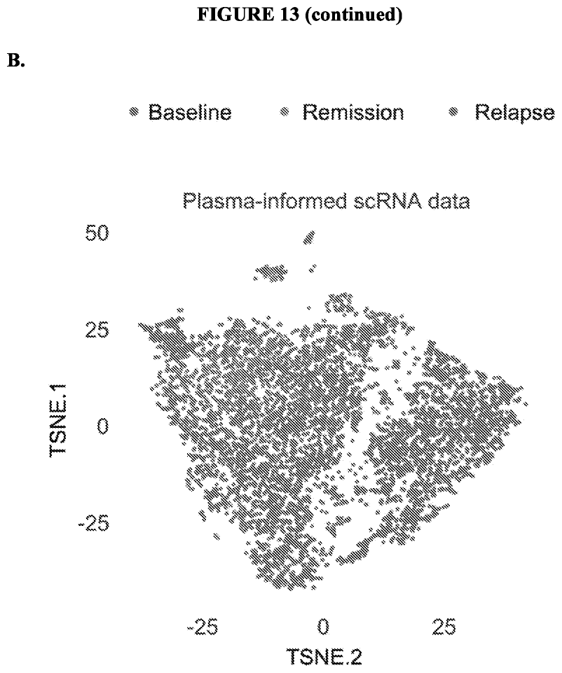

Six serial plasma samples from two AML patients undergoing therapy (FIG. 12A) with the bromodomain inhibitor, molibresib, were analysed using the plasma cfDNA method. The inferred gene expression profiles generated using the plasma cfDNA method were compared with matched serial single cell RNA (scRNA) sequencing data from bone marrow. In these patients, plasma whole genome sequencing did not reveal evidence of genomic evolution through analysis of CNA at the time of progression (FIG. 12B). Surprisingly, however, clustering of the inferred gene expression at each time point determined using the plasma cfDNA method closely mirrored that of the matched bone marrow scRNA sequencing in both patients and identified distinct transcriptional changes at time of progression (FIG. 12C and FIG. 13). For example, in AML patient 2, analysis of the scRNA sequencing data had previously revealed the emergence of a Leukemia Stem Cell (LSC) gene expression signature at the time of progression. This LSC gene expression signature was readily detected from the data generated using the plasma cfDNA method (FIG. 12D). These data demonstrate that the plasma cfDNA method described herein is effective for identifying evidence of transcriptional adaptation and the emergence of gene signatures specific to therapeutic resistance.

Finally, the process of trans-differentiation or lineage switching has been increasingly recognised as a mechanism of therapeutic escape in which cells transition from one cell identity to another, particularly in cancers where there is a multipotent cell of origin (see, e.g., Marine et al., 2020, Nature Reviews Cancer, 20:743-756). An example of this phenomenon is in lung cancer where EGFR mutant NSCLC can show trans-differentiation to a small cell phenotype following exposure to EGFR targeted therapy in an attempt to bypass the therapeutic challenge (Oser et al., 2015, The Lancet Oncology, 16: e165-e172). To assess if the plasma cfDNA method was effective to detect this lineage switching, a patient with NSCLC who was found to have neuroendocrine transformation following EGFR tyrosine kinase inhibitor therapy was used as a case study (FIG. 12E). Standard whole genome sequencing analysis of ctDNA from this patient did reveal evidence of genomic evolution at the time of disease progression with marked new copy number alterations identified. However, these data did not provide any insights into the transcriptional adaptation that had occurred (FIG. 12F). In contrast, application of the cfDNA method on samples taken at the time of disease progression revealed enrichment of key genes associated with the neuroendocrine transformation, including synaptophysin, CAM 5.2, and AE1.3, which were subsequently confirmed to be overexpressed via immunohistochemistry on the corresponding tumour biopsy (FIGS. 12G and 12H). These findings illustrate the strength of coupling both genomic and transcriptional analyses from ctDNA to characterise and understand the mechanisms by which cancers can evade therapeutic pressure.

### CONCLUSION

Collectively, these data demonstrate that cfDNA may be used to accurately and sensitively determine gene expression. The methods described herein have been reduced to practice in methods for determining the likelihood of a subject having cancer, across multiple cancer types, including both solid and haematological tumours. On this basis, integrated monitoring of non-genomic changes occurring in cancer can provide additional and more comprehensive information about disease evolution useful in informing or predicting response to cancer therapies.

Importantly, the methods disclosed herein are enabled to detect changes in disease status before clinicopathological relapse by comparing gene expression signatures of plasma samples taken before, during and/or after treatment. The methods disclosed herein are minimally invasive and both sensitive and accurate enough to track the cancer transcriptome in vivo to provide greater understanding into cancer evolution, thereby providing improved monitoring of disease progression and therapy response in patients, and stratifying patients for personalised therapies with a higher likelihood of response.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method for determining a level of gene expression from cell-free DNA (cfDNA), the method comprising:
a. providing a sample obtained from a subject, wherein the sample comprises fragments of cfDNA;
b. generating a plurality of sequence reads by sequencing the fragments of cfDNA, wherein the resulting plurality of sequence reads correspond to fragments of cfDNA of variable lengths;
c. aligning the plurality of sequence reads generated in step (b) with a reference genome;
d. identifying sets of sequence reads from step (c) that align to genomic regions of the reference genome comprising a gene transcriptional start site (TSS), wherein each set of sequence reads aligned to a genomic region of the reference genome corresponds to a single gene;
e. determining the read depth of the sets of sequence reads identified in step (d), wherein read depth is the number of unique sequence reads that align to each genomic region; and
f. generating a corrected read depth based on the read depth determined in step (e), a flanking region read depth and the total number of sequence reads in the sample, wherein the flanking region read depth is the number of unique sequence reads that align with the flanking region, wherein the flanking region comprises the centre of the nucleosome depleted region (NDR) for each TSS +/−5000 bp, wherein the corrected read depth corresponds to the level of gene expression for each gene, wherein the genes with the lowest corrected read depth have the highest gene expression, and wherein the genes with the highest corrected read depth have the lowest gene expression.

2. The method of claim 1, wherein the sample is selected from the group consisting of whole blood, serum and plasma.

3. The method of claim 2, wherein the sample is plasma.

4. The method of any one of claims 1 to 3, wherein the plurality of sequence reads is generated by low coverage whole-genome sequencing (LC-WGS).

5. The method of claim 4, wherein the LC-WGS results in between about 0.5× to about 20× sequencing coverage.

6. The method of any one of claims 1 to 5, wherein the reference genome is human reference genome assembly GRCh38 (hg38).

7. The method of any one of claims 1 to 6, wherein the sequence reads are at least 50 bp in length.

8. The method of claim 7, wherein the sequence reads are from about 80 bp to about 550 bp in length.

9. The method of any one of claims 1 to 8, each sequence read corresponds to a fragment of cfDNA comprising one or both of: (i) at least one nucleosome; and (ii) all or part of a DNA linker sequence adjacent to the at least one nucleosome.

10. The method of claim 9, wherein following the alignment in step (c), the sequence reads are trimmed to remove all or part of the DNA linker sequence.

11. The method of claim 10, wherein the alignment of the trimmed sequence reads to the reference genome is adjusted to generate a new start coordinate.

12. The method of any one of claims 1 to 11, wherein the genomic region comprising the TSS comprises positions +/−1000 with respect to the TSS.

13. The method of any one of claims 1 to 12, wherein the genomic region comprising the TSS comprises positions −150 bp to +50 bp with respect to the TSS.

14. The method of any one of claims 1 to 13, wherein the corrected read depth is the counts per million for each gene (CPMᵢ).

15. The method of any one of claims 1 to 14, wherein the sample comprises fragments of cell-free tumour DNA (ctDNA).

16. The method of any one of claims 1 to 15, further comprising:
g. repeating steps (b) to (f) with one or more additional samples obtained from the subject at a subsequent time point(s); and
h. comparing the level of gene expression for each gene determined in step (g) with the first sample to evaluate whether there has been a change in gene expression over time.

17. A method for determining the likelihood that a subject has cancer, the method comprising:
a. providing a sample obtained from the subject, wherein the sample comprises fragments of cfDNA;
b. determining the level of gene expression of one or more genes in the sample according to the method of any one of claims 1 to 15;
c. comparing the level of gene expression determined in step (b) with a reference level of gene expression for the one or more genes; and
d. based on the comparison in step (c), determining the likelihood that the subject has cancer.

18. The method of claim 17, wherein the reference level is a level of expression of the one or more genes that is predetermined from a sample obtained from one or more healthy subjects.

19. The method of claim 17, wherein the reference level is a level of expression of the one or more genes that is predetermined from a sample obtained from one or more subjects having cancer.

20. The method of any one of claims 17 to 19, wherein the one or more genes that is characteristic of cancer type.

21. The method of any one of claims 17 to 20, wherein the one or more genes that is characteristic of a cancer-associated pathway.

22. The method of any one of claims 17 to 21, wherein the one or more genes are differentially expressed in cancer relative to healthy controls.

23. A method for the treatment of a subject with cancer, the method comprising:
a. providing a sample obtained from the subject, wherein the sample comprises fragments of cfDNA;
b. determining the likelihood that a subject has cancer according to the method of any one of claims 17 to 22; and
c. where based on the determination in step (b) the subject has a high likelihood of having cancer, treating the subject with a treatment for said cancer.

24. The method of any one of claims 17 to 23, wherein the cancer is a haematological malignancy.

25. The method of claim 24, wherein the haematological malignancy is selected from MDS and AML.

26. The method of any one of claims 17 to 23, wherein the cancer is a solid tumour.

27. The method of claim 2, wherein the solid tumour is selected from the group consisting of breast cancer, lung cancer and melanoma.

28. The method of claim 23, wherein the cancer is MDS, and wherein the treatment is selected from the group consisting of a DNA hypomethylating agent, a thrombopoiesis-stimulating agent, and combinations of the foregoing.

29. The method of claim 23, wherein the cancer is AML, and wherein the treatment is a bromodomain inhibitor.

30. The method of claim 23, wherein the solid tumour is breast cancer, and wherein the treatment is selected from the group consisting of palbociclib, letrozole, and combinations of the foregoing.

31. The method of claim 23, wherein the solid tumour is melanoma and the treatment is selected from the group consisting of a MAPK inhibitor, immunotherapy, and combinations of the foregoing.

32. The method of claim 23, wherein the solid tumour is lung cancer, and wherein the treatment is a tyrosine kinase inhibitor.

33. A method for monitoring disease status in a subject having cancer, the method comprising:
a. providing a first sample obtained from the subject, wherein the first sample comprises fragments of cfDNA;
b. determining the level of gene expression of one or more genes in the first sample according to the method of any one of claims 1 to 15;
c. repeating steps (a) and (b) with one or more additional samples obtained from the subject at a subsequent time point(s);
d. determining the tumour fraction (%) for the first sample and the one or more additional samples;
e. normalising the level of gene expression in each sample determined in steps (b) and (c) based on the tumour fraction; and
f. comparing the normalised level of gene expression for each gene in the first sample with the normalised level of gene expression for each gene in the one or more additional samples to evaluate whether there has been a change in gene expression over time.

34. The method of claim 33, wherein the first sample is a baseline sample obtained from the subject prior to the commencement of a treatment for the cancer.

35. The method of claim 33 or claim 34, wherein the additional samples are obtained from the subject at a subsequent time point selected from the group consisting of during treatment, after treatment, and both during and after treatment.

36. The method of claim 35, wherein based on the comparison in step (f), the disease status of the subject is selected from responsive to the treatment and resistant to the treatment.

37. The method of claim 33 or claim 34, wherein based on the comparison in step (f), the disease status of the subject is selected from remission and relapse.
